(19) United States
(12) Patent Application Publication
(10) Pub. No.: US 2006/0252943 A1
(43) Pub. Date: Nov. 9, 2006

(54) Chemical process

(76) Inventors: Amogh Boloor, Houston, TX (US); Mui Cheung, Durham, NC (US); Jeffrey Alan Stafford, San Diego, CA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/518,349

(22) PCT Filed: Jun. 17, 2003
(86) PCT No.: PCT/US03/19211

§ 371 Date: Aug. 23, 2005

Related U.S. Application Data
(60) Provisional application No. 60/389,349, filed on Jun. 17, 2002.

(51) Int. Cl. C07D 231/56

(52) U.S. Cl. 548/361.1

(57) ABSTRACT

A process for the preparation of pyrimidine derivatives, which are useful as VEGFR2 inhibitors, is described herein. The described invention also includes pyrimidine derivatives as well as methods of using the same in the treatment of hyperproliferative diseases.

### BACKGROUND OF THE INVENTION

The present invention relates to pyrimidine derivatives, salts and solvates thereof as well as a process for preparing the same. In particular, the present invention relates to diamino substituted pyrimidines, anhydrous, hydrated and salt forms thereof, as well as processes for preparing the same.

The process of angiogenesis is the development of new blood vessels from pre-existing vasculature. Normal angiogenesis is active during tissue growth from embryonic development through maturity and then enters a period of relative quiescence during adulthood. Normal angiogenesis is also activated during wound healing, and at certain stages of the female reproductive cycle. Inappropriate or pathological angiogenesis has been associated with several disease states including various retinopathies, ischemic disease, atherosclerosis, chronic inflammatory disorders, and cancer. The role of angiogenesis in disease states is discussed, for instance, in Fan et al, Trends in Pharmacol Sci. 16:54-66; Shawver et al, DDT Vol. 2, No. 2 February 1997; Folkmann, 1995, Nature Medicine 1:27-31.

Central to the process of angiogenesis is vascular endothelial growth factor (VEGF) and it's receptors, termed vascular endothelial growth factor receptor(s) (VEGFRs). The roles VEGF and VEGFRs play in the vascularization of solid tumors, progression of hematopoietic cancers and modulation of vascular permeability have drawn great interest in the scientific community. VEGF is a polypeptide, which has been linked to inappropriate or pathological angiogenesis (Pinedo, H. M. et al The Oncologist, Vol. 5, No. 90001, 1-2, Apr. 2000). VEGFR(s) are protein tyrosine kinases (PTKs) that catalyze the phosphorylation of specific tyrosine residues in proteins that are involved in the regulation of cell growth, differentiation, and survival. (A. F. Wilks, Progress in Growth Factor Research, 1990, 2, 97-111; S. A. Courtneidge, Dev. Supp.l, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401).

Of particular interest is VEGFR2, which is a transmembrane receptor PTK expressed primarily in endothelial cells. Activation of VEGFR-2 by VEGF is a critical step in the signal transduction pathway that initiates tumor angiogenesis. VEGF expression may be constitutive to tumor cells and can also be upregulated in response to certain stimuli. One such stimulus is hypoxia, where VEGF expression is upregulated in both tumor and associated host tissues. The VEGF ligand activates VEGFR2 by binding to its extracellular VEGF binding site. This leads to receptor dimerization of VEGFRs and autophosphorylation of tyrosine residues at the intracellular kinase domain of VEGFR2. The kinase domain operates to transfer a phosphate from ATP to the tyrosine residues, thus providing binding sites for signaling proteins downstream of VEGFR-2 leading ultimately to angiogenesis. (Ferrara and Davis-Smyth, Endocrine Reviews, 18(1):4-25, 1997; McMahon, G., The Oncologist, Vol. 5, No. 90001, 3-10, Apr. 2000.)

Consequently, antagonism of the VEGFR2 kinase domain would block phosphorylation of tyrosine residues and serve to disrupt initiation of angiogenesis. Specifically, inhibition at the ATP binding site of the VEGFR2 kinase domain would prevent binding of ATP and prevent phosphorylation of tyrosine residues. Such disruption of the pro-angiogenesis signal transduction pathway associated with VEGFR2 should therefore inhibit tumor angiogenesis and thereby provide a potent treatment for cancer or other disorders associated with inappropriate angiogenesis.

The present inventors have discovered diamino substituted pyrimidines, salts and solvates thereof as well as processes for making the same. Such pyrimidine derivatives are inhibitors of VEGFR2 activity and are useful in the treatment of disorders, including cancer, associated with inappropriate angiogenesis.

### BRIEF SUMMARY OF THE INVENTION

In one aspect of the present invention, there is provided a process for preparing a compound of formula (R), 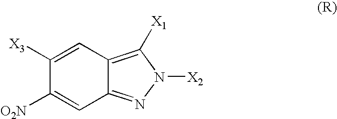
comprising the step of:

reacting a compound of formula (Q)

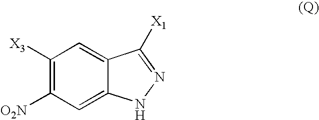

with an alkylating agent,

wherein

X₁ is hydrogen, C₁-C₄alkyl, C₁-C₄haloalkyl, or C₁-C₄hydroxyalkyl;
X₂ is C₁-C₄alkyl, C₁-C₄haloalkyl, or aralkyl; and
X₃ is hydrogen or halogen.

In a second aspect of the present invention, there is provided a process for preparing a compound of formula (I) 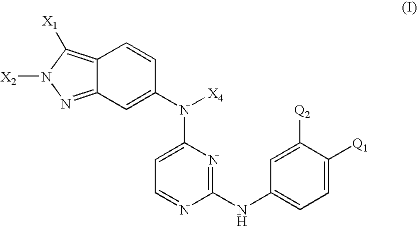
comprising the step of:

reacting a compound of formula (Q′) 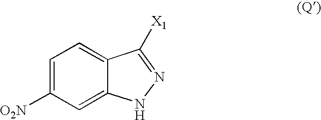
with an alkylating agent to prepare a compound of formula (R′), 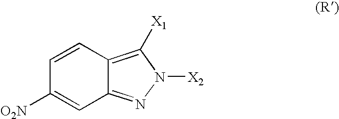
wherein:
X₁ is hydrogen or C₁-C₄alkyl;
X₂ is C₁-C₄alkyl or benzyl;
X₄ is hydrogen or C₁-C₄alkyl;
Q₁ is A¹ or A²;
Q₂is A¹ when Q₁ is A² and Q₂ is A² when Q₁ is A¹;
wherein
   A¹ is hydrogen, halogen, C₁-C₃alkyl, C₁-C₃haloalkyl, C₁-C₄alkoxy, and
   A² is the group defined by -(Z)ₘ-(Z¹)-(Z²), wherein
      Z is C(R′)(R″), where R′ and R″ are independently selected from —H or C₁-C₄ alkyl, or R′ and R″ together with the carbon to which they are attached form a C₃-C₇cycloalkyl group and m is 0, 1, 2, or 3;
      Z¹ is S(O)₂, S(O), or C(O); and
      Z² is C₁-C₄alkyl, NR¹R², aryl, arylamino, aralkyl, aralkoxy, or heteroaryl,
R¹ and R² are each independently selected from hydrogen, C₁-C₄alkyl, C₃-C₇ cycloalkyl, —S(O)₂R³, and —C(O)R³; and
R³ is C₁-C₄alkyl or C₃-C₇cycloalkyl.

In a third aspect of the present invention, there is provided a process for preparing a compound of formula (I) 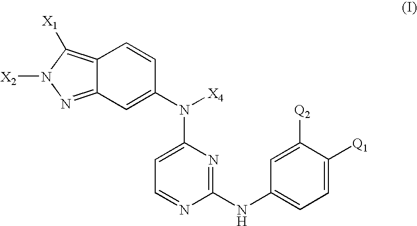
comprising the steps of:

(i) reacting a compound of formula (Q′)

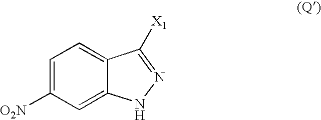

with an alkylating agent to prepare a compound of formula (R′), 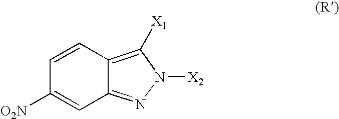
and

(ii) converting the compound of formula (R′) to the compound of formula (I), said converting step comprising condensation with a compound of formula (A′) and then a compound of formula (A″) 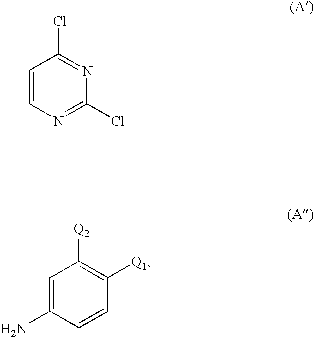
wherein:
X₁ is hydrogen or C₁-C₄alkyl;
X₂ is C₁-C₄alkyl or benzyl;
X₄ is hydrogen or C₁-C₄alkyl;
Q₁ is A¹ or A²;
Q₂ is A¹ when Q₁ is A² and Q₂ is A² when Q₁ is A¹;
wherein
   A¹ is hydrogen, halogen, C₁-C₃alkyl, C₁-C₃haloalkyl, C₁-C₄alkoxy, and
   A² is the group defined by -(Z)ₘ-(Z¹)-(Z²), wherein
      Z is C(R′)(R″), where R′ and R″ are independently selected from —H or C₁-C₄ alkyl, or R′ and R″ together with the carbon to which they are attached form a C₃-C₇ cycloalkyl group and m is 0, 1, 2, or 3;
      Z¹ is S(O)₂, S(O), or C(O); and
      Z² is C₁-C₄alkyl, NR¹R², aryl, arylamino, aralkyl, aralkoxy, or heteroaryl,
R¹ and R² are each independently selected from hydrogen, C₁-C₄alkyl, C₃-C₇ cycloalkyl, —S(O)₂R³, and —C(O)R³; and
R³ is C₁-C₄alkyl or C₃-C₇cycloalkyl.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “lower” refers to a group having between one and six carbons.

As used herein, the term “alkyl” refers to a straight or branched chain hydrocarbon having from one to twelve carbon atoms, optionally substituted with substituents selected from the group consisting of lower alkyl, lower alkoxy, lower alkylsulfanyl, lower alkylsulfenyl, lower alkylsulfonyl, oxo, mercapto, amino optionally substituted by alkyl, carboxy, carbamoyl optionally substituted by alkyl, aminosulfonyl optionally substituted by alkyl, nitro, or lower perfluoroalkyl, multiple degrees of substitution being allowed. Examples of “alkyl” as used herein include, but are not limited to, n-butyl, n-pentyl, isobutyl, and isopropyl, and the like.

As used herein, the term “C₁-C₄alkyl” refers to an alkyl group, as defined above, which contains at least 1, and at most 4, carbon atoms. Examples of “C₁-C₄ alkyl groups useful in the present invention include, but are not limited to, methyl, ethyl, propyl, isopropyl, isobutyl and n-butyl. In a like manner, the term “C₁-C₃alkyl” refers to an alkyl group, as defined above, which contains at least 1, and at most 3, carbon atoms respectively. Examples of “C₁-C₃ alkyl groups useful in the present invention include, methyl, ethyl, n-propyl and isopropyl.

As used herein the term “alkylene” refers to a straight or branched chain hydrocarbon radical having from one to ten carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of “alkylene” as used herein include, but are not limited to, methylene, ethylene, n-propylene, n-butylene, and the like.

As used herein, the term “C₁-C₄alkylene” refers to an alkylene group, as defined above, which contains at least 1, and at most 4, carbon atoms respectively. Examples of “C₁-C₄ alkylene” groups useful in the present invention include, but are not limited to, methylene, ethylene, n-propylene, and n-butylene.

As used herein, the terms “halogen” or “halo” refer to fluoro (—F), chloro (—Cl), bromo (—Br), or iodo (—I).

As used herein, the term “C₁-C₄haloalkyl” refers to a straight or branched chain hydrocarbon containing at least 1, and at most 4, carbon atoms substituted with at least one halogen, halogen being as defined herein. Examples of branched or straight chained “C₁-C₄ haloalkyl” groups useful in the present invention include, but are not limited to, methyl, ethyl, propyl, isopropyl, isobutyl and n-butyl substituted independently with one or more halogens, e.g., fluoro, chloro, bromo and iodo.

In a like manner, the term “C₁-C₃ haloalkyl” refers to a straight or branched chain hydrocarbon containing at least 1, and at most 3, carbon atoms respectively substituted with at least one halogen, halogen being as defined herein. Examples of branched or straight chained “C₁-C₃ haloalkyl” groups useful in the present invention include, but are not limited to, methyl, ethyl, n-propyl, and isopropyl substituted independently with one or more halogens, e.g., fluoro, chloro, bromo and iodo.

As used herein, the term “hydroxy” refers to the group —OH.

As used herein, the term “C₁-C₄ hydroxyalkyl” refers to a straight or branched chain hydrocarbon containing at least 1, and at most 4, carbon atoms substituted with at least one hydroxy, hydroxy being as defined herein. Examples of branched or straight chained “C₁-C₄ hydroxyalkyl” groups useful in the present invention include, but are not limited to, methyl, ethyl, propyl, isopropyl, isobutyl and n-butyl substituted independently with one or more hydroxy groups.

As used herein, the term “C₃-C₇ cycloalkyl” refers to a non-aromatic cyclic hydrocarbon ring having from three to seven carbon atoms, which optionally includes a C₁-C₄ alkylene linker through which it may be attached. Exemplary “C₃-C₇ cycloalkyl” groups include, but are not limited to, cyclopropyl, cyclobutyl, cyclopentyl, cyclohexyl and cycloheptyl.

As used herein, the term “heterocyclic” or the term “heterocyclyl” refers to a three to twelve-membered non-aromatic ring being saturated or having one or more degrees of unsaturation containing one or more heteroatomic substitutions selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, pyran, 1,4-dioxane, 1,3-dioxane, piperidine, pyrrolidine, morpholine, tetrahydrothiopyran, tetrahydrothiophene, and the like.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “aryl” groups include, but are not limited to, phenyl, 2-naphthyl, 1-naphthyl, biphenyl, as well as substituted derivatives thereof.

As used herein, the term “aralkyl” refers to an aryl or heteroaryl group, as defined herein including both unsubstituted and substituted versions thereof, attached through a lower alkylene linker, wherein lower alkylene is as defined herein. As used herein, the term “heteroaralkyl” is included within the scope of the term “aralkyl”. The term heteroaralkyl is defined as a heteroaryl group, as defined herein, attached through a lower alkylene linker, lower alkylene is as defined herein. Examples of “aralkyl”, including “heteroaralkyl”, include, but are not limited to, unsubstituted and substituted benzyl, phenylpropyl, 2-pyridinylmethyl, 4-pyridinylmethyl, 3-isoxazolylmethyl, 5-methyl-3-isoxazolylmethyl, 2-imidazoyly ethyl. The substituted versions, for instance substituted benzyl, are substituted with at least one of the groups recited as optional substituents in the aryl and heteroaryl definitions above.

As used herein, the term “arylamino” refers to an aryl or heteroaryl group, as defined herein, attached through an amino group —NR²—, wherein R² is as defined herein.

As used herein, the term “heteroaryl” refers to a monocyclic five to seven membered aromatic ring, or to a fused bicyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof.

As used herein, the term “alkoxy” refers to the group RₐO—, where Rₐ is alkyl as defined above and the term “C₁-C₂ alkoxy” refers to the group RₐO—, where Rₐ is C₁-C₂ alkyl as defined above.

As used herein, the term “haloalkoxy” refers to the group RₐO—, where Rₐ is haloalkyl as defined above and the term “C₁-C₂ haloalkoxy” refers to the group RₐO—, where Rₐ is C₁-C₂ haloalkyl as defined above.

As used herein the term “aralkoxy” refers to the group R_{b}RₐO—, where Rₐ is alkylene and R_{b} is aryl, both as defined above.

As used herein, the term “alkylsulfanyl” refers to the group RₐS—, where Rₐ is alkyl as defined above.

As used herein, the term “alkylsulfenyl” refers to the group RₐS(O)—, where Rₐ is alkyl as defined above.

As used herein, the term “alkylsulfonyl” refers to the group RₐSO₂—, where Rₐ is alkyl as defined above.

As used herein, the term “oxo” refers to the group ═O

As used herein, the term “mercapto” refers to the group —SH.

As used herein, the term “carboxy” refers to the group —COOH.

As used herein, the term “cyano” refers to the group —CN.

As used herein the term “cyanoalkyl” refers to the group —RₐCN wherein Rₐ is C₁-C₃ alkylene as defined above. Exemplary “cyanoalkyl” groups useful in the present invention include, but are not limited to, cyanomethyl, cyanoethyl, and cyanopropyl.

As used herein, the term “aminosulfonyl” refers to the group —SO₂NH₂.

As used herein, the term “carbamoyl” refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂— or —S(O₂).

As used herein, the term “acyl” refers to the group RₐC(O)—, where Rₐ is alkyl, cycloalkyl, or heterocyclyl as defined herein.

As used herein, the term “aroyl” refers to the group RₐC(O)—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent Examples of suitable pharmaceutically acceptable solvents include water, ethanol and acetic acid. Most preferably the solvent used is water.

The compounds of formula (I) may have the ability to crystallize in more than one form, a characteristic, which is known as polymorphism, and it is understood that such polymorphic forms (“polymorphs”) are within the scope of formula (I). Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. Accordingly, the compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted.

It is also noted that the compounds of Formula (I) may form tautomers. It is understood that all tautomers and mixtures of tautomers of the compounds of the compounds of formula (I) are included within the scope of the compounds of the present invention.

The present invention includes a process for preparing a compound of formula (R)

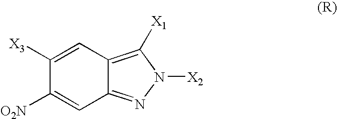

Generally, X₁ is hydrogen, C₁-C₄ alkyl, C₁-C₄ haloalkyl, or C₁-C₄ hydroxyalkyl; preferably X₁ is C₁-C₄alkyl; more preferably X₁ is methyl.

X₂ is C₁-C₄alkyl, C₁-C₄haloalkyl, or aralkyl; preferably X₂ is C₁-C₄alkyl or aralkyl. In one preferred embodiment, X₂ is benzyl. In another preferred embodiment, X₂ is methyl or ethyl, preferably methyl.

X₃ is hydrogen or halogen, preferably hydrogen.

In one embodiment, the compound of formula (R) is

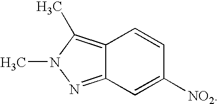

The compound of formula R is prepared by reacting a compound of formula (Q) 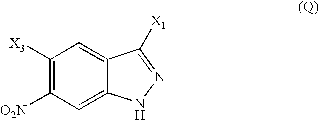
with an alkylating agent.

X₁ and X₃ of formula (Q) are as described above for formula (R).

Typically, the conditions for the N-2 alkylation of the compound of formula (Q) are any conditions suitable to effect such N-2 alkylation. Suitable alkylating agents are described for instance in Encyclopedia of Reagents for Organic Synthesis; Paquette, L. A., Ed.; John Wiley Et Sons, 1995. Examples include, but are not limited to, (1) reacting a compound of formula (Q) with a trialkyloxonium salt such as trimethyloxonium or triethyloxonium salts in organic solvents such as acetone, methyl acetate, ethyl acetate, and nitromethane, specifically, trimethyloxonium salts such as trimethyloxonium tetrafluoroborate and triethyloxonium salts such as trimethyloxonium tetrafluoroborate (Meerwein's salt) can be used as suitable alkylating agents (such trialkyloxonium salts are known in the art); (2) reacting a compound of formula (Q) with sulfuric acid and dimethyl sulfate in organic solvents such as DMSO and dichloromethane; and (3) reacting a compound of formula (Q) with trimethylorthoformate and boron trifluoride etherate (in situ generation of Borsch's reagent) in organic solvents such as dichloromethane.

The present invention also includes a process for preparing a compound of formula (I)

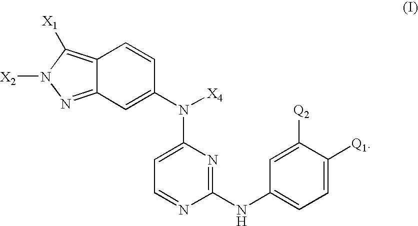

X₁ is hydrogen or C₁-C₄alkyl; preferably C₁-C₄alkyl; more preferably methyl.

X₂ is C₁-C₄ alkyl or benzyl; preferably methyl, ethyl or benzyl; more preferably methyl.

X₄ is hydrogen or C₁-C₄ alkyl; preferably methyl or ethyl, more preferably methyl.

Q₁ is A¹ or A² where Q₂ is A¹ when Q₁ is A² and Q₂ is A² when Q₁ is A¹; preferably Q₂ is A² when Q₁ is A¹, where A¹ is hydrogen, halogen, C₁-C₃ alkyl, C₁-C₃ haloalkyl, —O(C₁-C₄ alkyl), preferably A¹ is C₁-C₃ alkyl, C₁-C₃ haloalkyl, or —O(C₁-C₄ alkyl), more preferably A¹ is C₁-C₃ alkyl, most preferably methyl and A² is the group defined by -(Z)ₘ-(Z¹)-(Z²), wherein
Z is C(R′)(R″), where R′ and R″ are independently selected from —H or C₁-C₄alkyl, or R′ and R″ together with the carbon to which they are attached form a C₃-C₇cycloalkyl group and m is 0, 1, 2, or 3;
Z¹ is S(O)₂, S(O), or C(O); and
Z² is C₁-C₄alkyl, NR¹R², aryl, arylaminro, aralkyl, aralkoxy, or heteroaryl,
R¹ and R² are each independently selected from hydrogen, C₁-C₄alkyl, C₃-C₇cycloalkyl, —S(O)₂R³, and —C(O)R³; and
R³ is C₁-C₄alkyl or C₃-C₇cycloalkyl.

In one embodiment, Q₁ is A² and Q₂ is A¹, A¹ is hydrogen, m is 1 and A² is -(Z)ₘ-(Z¹)-(Z²); where Z is C(R′)(R″), where R′ and R″ are each hydrogen; Z¹ is S(O)₂, and Z² is C₁-C₄alkyl, preferably methyl or ethyl, more preferably methyl.

In another embodiment, Q₁ is A¹ and Q₂ is A², A¹ is C₁-C₄-alkyl, preferably methyl or ethyl, more preferably methyl, m is 0 and A² is -(Z¹)-(Z²); where Z¹ is S(O)₂, and Z² is NR¹R², where R¹ and R² are each independently selected from hydrogen, C₁-C₄alkyl, C₃-C₇ cycloalkyl, —S(O)₂R³, and —C(O)R³, where R³ is as defined above; preferably R¹ and R² are each independently hydrogen or methyl: preferably each of R¹ and R² is hydrogen.

In another embodiment, the process of preparing a compound of formula (I) includes the step of:

(i) reacting a compound of formula (Q′)

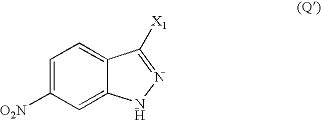

with an alkylating agent to prepare a compound of formula (R′),

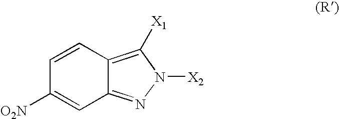

X₁, X₂, and the alkylating agent are as defined above.

Such process may further comprise a step (ii) wherein the compound of formula (R) is converted to a compound of formula (I) by condensation with a compound of formula (A′) and then a compound of formula (A″)

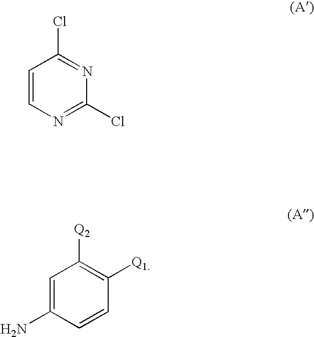

Q₁ and Q₂are as described above.

In a further embodiment, the process includes a further step (ii′) reducing the compound of formula (R′) to a compound of formula (R″):

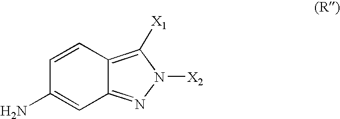

Such step (ii′) is typically performed before or concurrently with step (ii).

In a further embodiment, the process includes a further step (iii′) alkylating the compound of formula (S) to a compound of formula (T):

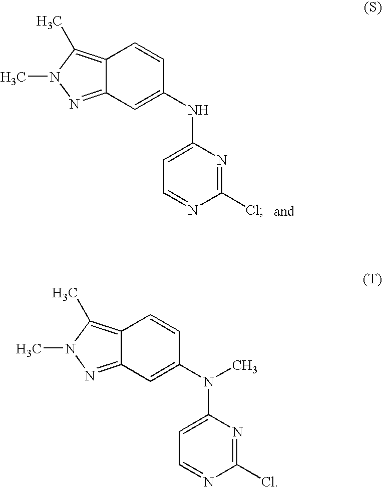

Such step (ii′) is typically performed before or concurrently with the second condensation step as illustrated in Scheme 1. The alkylation is performed using methods known in the art, see Encyclopedia of Reagents for Organic Synthesis; Paquette, L. A., Ed.; John Wiley Et Sons, 1995, and is further described in Scheme 1 and the Examples following.

In still another embodiment, the process includes a further step (iii) converting the compound of formula (I) into a salt and/or solvated form of the compound of formula (I).

Scheme 1 depicts one embodiment of a process for preparing compounds of Formula (I). A substituted 6-nitroindazole Q′ undergoes alkylation by an appropriate alkylating agent (see above) to provide the N2-alkylated nitroindazole R′. Reduction of the nitro group using standard conditions (e.g., SnCl₂, aqueous acid or 10% Pd/C, methanol, ammonium formate) followed by condensation with 2,4-dichloropyrimidine provides the chloropyrimidine S. Alkylation of the bisaryl amine nitrogen under appropriate alkylation conditions (e.g., MeI, Cs₂CO₃ or NaH, DMF) affords intermediate T. which undergoes subsequent condensation with an appropriately substituted aniline (A″) to provide the compound of Formula (I). X₁, X₂, X₄, Q₁ and Q₂ are as described above.

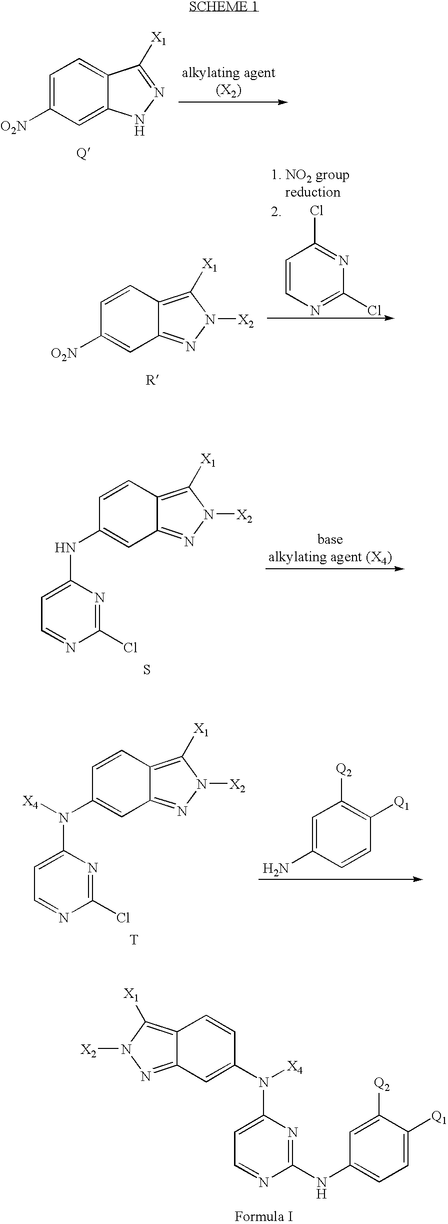

In another aspect of the present invention, there is provided a compound of Formula (I): 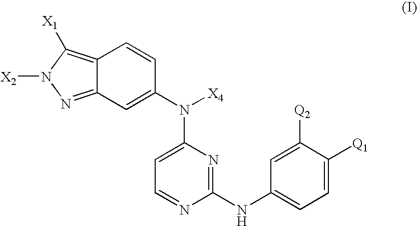
or a salt, solvate, or physiologically functional derivative thereof:
wherein:
X₁ is hydrogen, C₁-C₄alkyl, or C₁-C₄hydroxyalkyl;
X₂ is C₁-C₄alkyl or benzyl;
X₄ is hydrogen or C₁-C₄alkyl;
Q₁ is A¹ or A²;
Q₂ is A¹ when Q₁ is A²and Q₂ is A² when Q₁ is A¹;
wherein
   A¹ is hydrogen, halogen, C₁-C₃ alkyl, C₁-C₃ haloalkyl, —O(C₁-C₄alkyl), and
   A² is the group defined by -(Z)ₘ-(Z¹)-(Z²), wherein
      Z is C(R′)(R″), where R′ and R″ are independently selected from —H or C₁-C₄alkyl, or R′ and R″ together with the carbon to which they are attached form a C₃-C₇ cycloalkyl group and m is 0, 1, 2, or 3;
      Z¹ is S(O)₂, S(O), or C(O); and
      Z² is C₁-C₄alkyl, NR¹R², aryl, arylamino, aralkyl, aralkoxy, or heteroaryl,
R¹ and R² are each independently selected from hydrogen, C₁-C₄ alkyl, C₃-C₇ cycloalkyl, —S(O)₂R³, and —C(O)R³; and
R³ is C₁-C₄alkyl or C₃-C₇cycloalkyl.

In one embodiment, X₁ is hydrogen or C₁-C₄alkyl; preferably C₁-C₄alkyl; more preferably methyl.

In one embodiment, X₂ is C₁-C₄ alkyl or benzyl; preferably methyl, ethyl or benzyl; more preferably methyl.

In one embodiment, X₄ is hydrogen or C₁-C₄ alkyl; preferably methyl or ethyl; more preferably methyl.

In one embodiment, Q₁ is A¹ or A² where Q₂ is A¹ when Q₁ is A² and Q₂ is A² when Q₁ is A¹; preferably Q₂ is A² when Q₁ is A¹, where A¹ is hydrogen, halogen, C₁-C₃ alkyl, C₁-C₃ haloalkyl, —O(C₁-C₄ alkyl), preferably A¹ is C₁-C₃ alkyl, C₁-C₃ haloalkyl, or —O(C₁-C₄alkyl), more preferably A¹ is C₁-C₃ alkyl, most preferably methyl and A² is the group defined by -(Z)ₘ-(Z¹)-(Z²), wherein
Z is C(R′)(R″), where R′ and R″ are independently selected from —H or C₁-C₄alkyl, or R′ and R″ together with the carbon to which they are attached form a C₃-C₇cycloalkyl group and m is 0, 1, 2, or 3;
Z¹ is S(O)₂, S(O), or C(O); and
Z² is C₁-C₄alkyl, NR¹R², aryl, arylamino, aralkyl, aralkoxy, or heteroaryl,
R¹ and R² are each independently selected from hydrogen, C₁-C₄alkyl, C₃-C₇ cycloalkyl, —S(O)₂R³, and —C(O)R³; and
R³ is C₁-C₄ alkyl or C₃-C₇ cycloalkyl.

In one embodiment, Q₁ is A² and Q₂ is A¹, A¹ is hydrogen, m is 1 and A² is -(Z)m-(Z¹)-(Z²); where Z is C(R′)(R″), where R′ and R″ are each hydrogen; Z¹ is S(O)₂, and Z² is C₁-C₄alkyl, preferably methyl or ethyl, more preferably methyl.

In another embodiment, Q₁ is A¹ and Q₂ is A², A¹ is C₁-C₄alkyl, preferably methyl or ethyl, more preferably methyl, m is 0 and A² is -(Z¹)-(Z²); where Z¹ is S(O)₂, and Z² is NR¹R², where R¹ and R² are each independently selected from hydrogen, C₁-C₄alkyl, C₃-C₇ cycloalkyl, —S(O)₂R³, and —C(O)R³, where R³ is as defined above; preferably R¹ and R² are each independently hydrogen or methyl; preferably each of R¹ and R² is hydrogen.

In another embodiment, there is provided compounds useful as intermediates in the preparation of compounds of formula (I):

### 2,3-dimethyl-6-nitro-2H-indazole

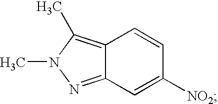

### 2,3-dimethyl-6-amino-2H-indazole

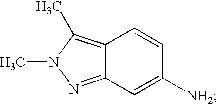

### N-(2-chloropyrimidin-4-yl)-2,3-dimethyl-2H-indazol-6-amine

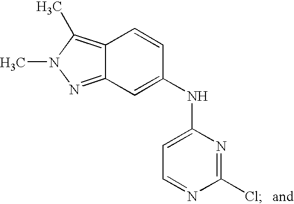

### N-(2-chloropyrimidin-4-yl)-N,2,3-trimethyl-2H-indazol-6-amine

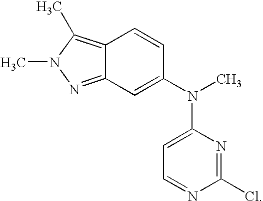

While it is possible that, for use in therapy, therapeutically effective amounts of a compound of formula (I), as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates, and physiological functional derivatives thereof, with one or more pharmaceutically acceptable carriers, diluents, or excipients.

Pharmaceutical formulations may be presented in unit dose forms containing a predetermined amount of active ingredient per unit dose. Such a unit may contain, for example, 0.5 mg to 1 g, preferably 1 mg to 700 mg, of a compound of the formula (I) depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 50 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of the present invention and their salts and solvates, and physiologically functional derivatives thereof, may be employed alone or in combination with other therapeutic agents for the treatment of the above-mentioned conditions. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of formula (I) or a pharmaceutically acceptable salt or solvate thereof, or a physiologically functional derivative thereof, and the use of at least one other cancer treatment method. Preferably, combination therapies according to the present invention comprise the administration of at least one compound of formula (I) or a pharmaceutically acceptable salt or solvate thereof, or a physiologically functional derivative thereof, and at least one other pharmaceutically active agent, preferably an anti-neoplastic agent. The compound(s) of formula (I) and the other pharmaceutically active agent(s) may be administered together or separately and, when administered separately this may occur simultaneously or sequentially in any order. The amounts of the compound(s) of formula (I) and the other pharmaceutically active agent(s) and the relative timings of administration will be selected in order to achieve the desired combined therapeutic effect.

The compounds of the Formula (I) or salts, solvates, or physiologically functional derivatives thereof and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent The administration in combination of a compound of formula (I) or salts, solvates, or physiologically functional derivatives thereof with other anti-neoplastic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compounds and salts, solvates or physiologically functional derivatives thereof of formula I include the following:

(1) cell cycle specific anti-neoplastic agents including, but not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine ; antimetabolites such as allopurinol, fludurabine, methotrexate, cladrabine, cytarabine, mercaptopurine and thioguanine; and camptothecins such as 9-amino camptothecin, irinotecan, CPT-11 and the various optical forms of 7-(4-methylpiperazino-methylene)-10,11-ethylenedioxy-20-camptothecin;

(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumour antibiotics such as doxorubicin, daunomycin, epirubicin, idarubicin, mitomycin-C, dacttinomycin and mithramycin; and platinum coordination complexes such as cisplatin, carboplatin, and oxaliplatin; and

(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamide, bicalutamide, and cyproterone acetate; LHRH agonists and antagagonists such as goserelin acetate and luprolide, testosterone 5α-dihydroreductase inhibitors such as finasteride; metalloproteinase inhibitors such as marimastat; antiprogestogens; urokinase plasminogen activator receptor function inhibitors; cyclooxygenase type 2 (COX-2) inhibitors such as celecoxib; other angiogenic inhibiting agents such as VEGFR inhibitors other than those described herein and TIE-2 inhibitors; growth factor function inhibitors such as inhibitors of the functions of hepatocyte growth factor; erb-B2, erb-B4, epidermal growth factor receptor (EGFr), platelet derived growth factor receptor (PDGFr), fibroblast growth factor receptor (FGFr), vascular endothelial growth factor receptor (VEGFR) other than those described in the present invention, and TIE-2; and other tyrosine kinase inhibitors such as cyclin dependent inhibitors such as CDK2 and CDK4 inhibitors.

The compounds of formula (I) and salts, solvates and physiological functional derivatives thereof, are believed to have anticancer activity as a result of inhibition of the protein kinase VEGFR2 and its effect on selected cell lines whose growth is dependent on VEGFR2 protein kinase activity.

The present invention thus also provides compounds of formula (I) and pharmaceutically acceptable salts or solvates thereof, or physiologically functional derivatives thereof, for use in medical therapy, and particularly in the treatment of disorders mediated by inappropriate VEGFR2 activity.

The inappropriate VEGFR2 activity referred to herein is any VEGFR2 activity that deviates from the normal VEGFR2 activity expected in a particular mammalian subject. Inappropriate VEGFR2 activity may take the form of, for instance, an abnormal increase in activity, or an aberration in the timing and or control of VEGFR2 activity. Such inappropriate activity may result then, for example, from overexpression or mutation of the protein kinase or ligand leading to inappropriate or uncontrolled activation of the receptor. Furthermore, it is also understood that unwanted VEGFR2 activity may reside in an abnormal source, such as a malignancy. That is, the level of VEGFR2 activity does not have to be abnormal to be considered inappropriate, rather the activity derives from an abnormal source. In a like manner, the inappropriate angiogenesis referred to herein is any angiogenic activity that deviates from the normal angiogenic activity expected in a particular mammalian subject. Inappropriate angiogenesis may take the form of, for instance, an abnormal increase in activity, or an aberration in the timing and or control of angiogenic activity. Such inappropriate activity may result then, for example, from overexpression or mutation of a protein kinase or ligand leading to inappropriate or uncontrolled activation of angiogenesis. Furthermore, it is also understood that unwanted angiogenic activity may reside in an abnormal source, such as a malignancy. That is, the level of angiogenic activity does not have to be abnormal to be considered inappropriate, rather the activity derives from an abnormal source.

The present invention is directed to methods of regulating, modulating, or inhibiting VEGFR2 for the prevention and/or treatment of disorders related to unregulated VEGFR2 activity. In particular, the compounds of the present invention can also be used in the treatment of certain forms of cancer. Furthermore, the compounds of the present invention can be used to provide additive or synergistic effects with certain existing cancer chemotherapies and radiation, and/or be used to restore effectiveness of certain existing cancer chemotherapies and radiation.

The compounds of the present invention are also useful in the treatment of one or more diseases afflicting mammals which are characterized by cellular proliferation in the area of disorders associated with neo-vascularization and/or vascular permeability including blood vessel proliferative disorders including arthritis and restenosis; fibrotic disorders including hepatic cirrhosis and atherosclerosis; mesangial cell proliferative disorders include glomerulonephritis, diabetic nephropathy, malignant nephrosclerosis, thrombotic microangiopathy syndromes, proliferative retinopathies, organ transplant rejection and glomerulopathies; and metabolic disorders include psoriasis, diabetes mellitus, chronic wound healing, inflammation and neurodegenerative diseases.

A further aspect of the invention provides a method of treatment of a mammal suffering from a disorder mediated by inappropriate VEGFR2 activity, including susceptible malignancies, which includes administering to said subject an effective amount of a compound of formula (I) or a pharmaceutically acceptable salt, solvate, or a physiologically functional derivative thereof. In a preferred embodiment, the disorder is cancer.

A further aspect of the invention provides a method of treatment of a mammal suffering from cancer, which includes administering to said subject an effective amount of a compound of formula (I) or a pharmaceutically acceptable salt or solvate thereof, or a physiologically functional derivative thereof.

A further aspect of the present invention provides the use of a compound of formula (I), or a pharmaceutically acceptable salt or solvate thereof, or a physiologically functional derivative thereof, in the preparation of a medicament for the treatment of a disorder characterized by inappropriate VEGFR2 activity. In a preferred embodiment, the disorder is cancer.

A further aspect of the present invention provides the use of a compound of formula (I), or a pharmaceutically acceptable salt or solvate thereof, or a physiologically functional derivative thereof, in the preparation of a medicament for the treatment of cancer and malignant tumours.

The mammal requiring treatment with a compound of the present invention is typically a human being.

In another embodiment, therapeutically effective amounts of the compounds of formula (I) or salts, solvates or physiologically derived derivatives thereof and agents which inhibit growth factor receptor function may be administered in combination to a mammal for treatment of a disorder mediated by inappropriate VEGFR2 activity, for instance in the treatment of cancer. Such growth factor receptors include, for example, EGFR, FGFR, PDGFR, erbB2, erbB4, VEGFR, and/or TIE-2. Growth factor receptors and agents that inhibit growth factor receptor function are described, for instance, in Kath, John C., Exp. Opin. Ther. Patents (2000) 10(6):803-818 and in Shawver et al DDT Vol 2, No. 2 February 1997.

The compounds of the Formula (I) or salts, solvates, or physiologically functional derivatives thereof and the agent for inhibiting growth factor receptor function may be employed in combination concomitantly or sequentially in any therapeutically appropriate combination. The combination may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one is administered first and the other second or vice versa. Such sequential administration may be close in time or remote in time.

In another aspect of the present invention, there is provided a method of treating a disorder in a mnammal, said disorder being mediated by inappropriate angiogenesis, including: administering to said mammal a therapeutically effective amount of a compound of formula (I), or a salt, solvate or physiologically functional derivative thereof. In one embodiment, the inappropriate angiogenic activity is due to at least one of inappropriate VEGFR1, VEGFR2, VEGFR3, or TIE-2 activity. In another embodiment, the inappropriate angiogenesis is due to inappropriate VEGFR2 and TIE-2 activity. In a further embodiment, the method further includes administering a therapeutically effective amount of a TIE-2 inhibitor along with the compounds of formula (I) or salts, solvates or physiologically functional derivatives thereof. Preferably the disorder is cancer.

In another aspect of the present invention, there is provided the use of a compound of formula (I), or a salt, solvate or physiologically functional derivative thereof in the preparation of a medicament for use in treating a disorder in a mammal, said disorder being characterized by inappropriate angiogenesis. In one embodiment, the inappropriate angiogenic activity is due to at least one of inappropriate VEGFR1, VEGFR2, VEGFR3 or TIE-2 activity. In another embodiment, the inappropriate angiogenic activity is due to inappropriate VEGFR2 and TIE-2 activity. In a further embodiment, the use further includes use of a TIE-2 inhibitor to prepare said medicament.

The combination of a compound of formula (I) or salts, solvates, or physiologically functional derivatives with a TIE-2 inhibitor may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one is administered first and the other second or vice versa. Such sequential administration may be close in time or remote in time.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. Generally, the following schemes are illustrated using compounds of formula (I), but it is recognized that such schemes are easily adaptable by the skilled artisan to prepare compounds of formula (I), including compounds of formula (I′) and (I″). It is also recognized that in all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley Et Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-lnterscience, 1994).

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); RT (room temperature);
min (minutes); h (hours);
mp (melting point); TLC (thin layer chromatography);
Tᵣ (retention time); RP (reverse phase);
MeOH (methanol); I-PrOH (isopropanol);
TEA (triethylamine); TFA (trifluoroacetic acid);
TFAA (trifluoroacetic anhydride); THF (tetrahydrofuran);
DMSO (dimethylsulfoxide); EtOAc (ethyl acetate);
DME (1,2-dimethoxyethane); DCM (dichloromethane);
DCE (dichloroethane); DMF (N,N-dimethylformamide);
DMPU (N,N′-dimethylpropyleneurea); (CDl (1,1-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid); EDC (ethylcarbodiimide hydrochloride);
BOC (tert-butyloxycarbonyl); FMOC (9-fluorenylmethoxycarbonyl);
DCC (dicyclohexylcarbodiimide); CBZ (benzyloxyca rbonyl);
Ac (acetyl); atm (atmosphere);
TMSE (2-(trimethylsilyl)ethyl); TMS (trimethylsilyl);
TIPS (triisopropylsilyl); TBS (t-butyidimethylsilyl);
DMAP (4-dimethylaminopyridine); Me (methyl);
OMe (methoxy); Et (ethyl);
HPLC (high pressure liquid chromatography);
BOP (bis(2-oxo-3-oxazolidinyl)phosphinic chloride);
TBAF (tetra-n-butylammonium fluoride);
Et (ethyl); tBu (tert-butyl).

All references to ether are to diethyl ether; brine refers to a saturated aqueous solution of NaCI. Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX₅₀₅HA, JOEL SX-102, or a SCIEX-APliii spectrometer; high resolution MS were obtained using a JOEL SX-102A spectrometer. All mass spectra were taken under electrospray ionization (ESI), chemical ionization (CI), electron impact (El) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

The following examples describe the syntheses of intermediates particularly useful in the synthesis of compounds of Formula (I):

### Intermediate Example 1

### Preparation of 2,3-dimethyl-6-nitro-2H-indozole

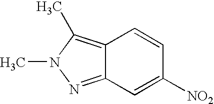 Procedure 1:

To a stirred solution of 18.5 g (0.11 mol) of 3-methyl-6-nitro-1H-indazole in 350 ml acetone, at room temperature, was added 20 g (0.14 mol) of trimethyloxonium tetraflouroborate. After the solution was allowed to stir under argon for 3 hours, the solvent was removed under reduced pressure. To the resulting solid was added saturated aqueous NaHCO₃ (600 mL) and a 4:1 mixture of chloroform-isopropanol (200 ml), the mixture was agitated and the layers were separated. The aqueous phase was washed with additional chloroform: isopropanol (4×200 mL) and the combined organic phase was dried (Na₂SO₄). Filtration and removal of solvent gave a tan solid. The solid was washed with ether (200 mL) to afford 2,3-dimethyl-6-nitro-2H-indazole as a yellow solid (15.85 g, 73%). ¹H NMR (300 MHz, DMSO-d₆) δ 8.51 (s, 1H), 7.94 (d, J=9.1 Hz, 1H). 7.73 (d, J=8.9 Hz, 1H), 4.14 (s, 3H), 2.67 (s, 3H). MS (ES+, m/z) 192 (M+H).

Procedure 2:

Trimethyl orthoformate (11 mmol, 1.17 g) was added over a 2 min period to a solution of boron trifluoride etherate (12.5 mmol, 1.77 g in methylene chloride (2.0 mL) which had been cooled to −30° C. The mixture was warmed to 0° C. for 15 min and was then cooled to −70° C. The nitro indazole (10 mmol, 1.77 g) was slurried in methylene chloride (30 mL) and was added all at once to the cooled mixture. The mixture was stirred at −70° C. for 15 min and at ambient temperature for 17 h. After 17 h the mixture was red and heterogeneous. The reaction mixture was quenched with saturated sodium bicarbonate solution (20 mL) and the organic layer separated. The aqueous layer was extracted with methylene chloride (30 mL). The methylene chloride layers were combined and extracted with water (30 mL). The methylene chloride layer was distilled under reduced pressure until ˜10 mL remained. Propanol (10 mL) was added and the remainder of the methylene chloride removed under reduced pressure, resulting in a yellow slurry. The product was isolated by filtration to give 2,3-dimethyl-6-nitro-2H-indazole (65%, 7 mmol, 1.25 g) as a light yellow powder. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

Procedure 3:

In a 25 ml round bottom flask 3-methyl-6-nitroindazole (7.27 mmol, 1.28 g) was dissolved with stirring in DMSO (4.0 mL) and was treated with concentrated sulfuric acid (7.27 mmol, 0.73 g) to yield a thick slurry. The slurry was treated with dimethyl sulfate (21.1 mmol, 2.66 g). The mixture was heated under nitrogen at 50° C. for 72 h. After 72 h a thick yellow slurry was obtained. The slurry was cooled and was slowly treated with saturated sodium bicarbonate solution (10 mL). The mixture was extracted with methylene chloride (2×20 mL). The methylene chloride layers were combined and back extracted with water (20 mL). The methylene chloride layer was treated with propanol (10 mL) and the methylene chloride was removed by distillation under reduced pressure. The solid was isolated by filtration and the yellow solid washed with heptane (5 mL) and air-dried. The 2,3-dimethyl-6-nitro-2H-indazole product (70%, 0.97 g) was obtained as a light yellow solid. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

Procedure 4:

Into a 250 mL 3-necked round bottom flask was placed 3-methyl-6-nitro-1H-indazole sulfuric acid salt (5.0 g, 18.2 mmol) and methylene chloride (25 mL). The mixture was stirred at 25° C. and was treated with DMSO (5 mL). Dimethyl sulfate (6.7 g, 5.0 mL, 53.0 mmol) was added via syringe and the reaction was heated at reflux in a 70° C bath. After 7 h HPLC analysis showed 9% starting material. At this point heating was stopped and the workup begun. Saturated sodium bicarbonate solution (35 mL) was added to the reaction mixture at RT. The layers were allowed to separate and the aqueous layer was extracted with methylene chloride (25 mL). The methylene chloride layers were combined and washed with water (2×25 mL). The methylene chloride layer was distilled under reduced pressure until half the volume was removed. Propanol (25 mL) was added and distillation under reduced pressure was continued until all the methylene chloride had been removed. This yielded a yellow slurry, which was allowed to stir at 25° C. for 1 h. The product was isolated via filtration and the resulting yellow solid was washed with heptane (10 mL). This yielded 2,3-dimethyl-6-nitro-2H-indazole (70%, 2.43 g) as a yellow solid. ¹H NMR (300 MHz, DMSO-d₆) δ 8.51 (s, 1H), 7.94 (d, J=9.1 Hz, 1H), 7.73 (d, J=8.9 Hz, 1H), 4.14 (s, 3H), 2.67 (s, 3H). MS (ES+, m/z) 192 (M+H).

### Intermediate Example 2

### Preparation of 2,3-dimethyl-6-amino-2H-indazole

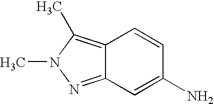 Procedure 1:

To a stirred solution of 2,3-dimethyl-6-nitro-2H-indazole (1.13 g) in 2-methoxyethyl ether (12 ml), at 0° C., was added a solution of 4.48 g of tin(II) chloride in 8.9 ml of concentrated HCl dropwise over 5 min. After the addition was complete, the ice bath was removed and the solution was allowed to stir for an additional 30 min. Approximately 40 ml of diethyl ether was added to reaction, resulting in precipitate formation. The resulting precipitate was isolated by filtration and washed with diethyl ether, and afforded a yellow solid (1.1 g, 95%), the HCl salt 2,3-dimethyl-2H-indazol-6-amine. ¹H NMR (300 MHz, DMSO-d₆) δ 7.77 (d, J=8.9 Hz, 1H), 7.18 (s, 1H), 7.88 (m, 1H), 4.04 (s, 3H), 2.61 (s, 3H). MS (ES+, m/z) 162 (M+H).

Procedure 2:

A 2-L 3-necked round bottom flask was fitted with nitrogen inlet and outlet and with mechanical stirring. A moderate nitrogen flow was initiated and the reactor was charged with 10% Pd/C (500% water wet, 6.0 g). Stirring was initiated and the reactor was charged with methanol (750 mL) and the product of Intermediate Example 1 (50 g). Ammonium formate (82.54 g) was dissolved in water (120 mL). The water solution of ammonium formate was added to the reaction solution at an addition rate, which kept the reaction temperature at or between 25 and 30° C. The reaction was allowed to proceed at 25° C. After 6 h the reaction was judged to be finished based on HPLC analysis. The mixture was filtered and the catalyst washed with methanol (50 mL). The methanol layers were combined and the solvent removed under reduced pressure. The residue was dissolved in water (200 mL) and was extracted with methylene chloride (3×250 mL). The methylene chloride layers were combined and solvent removed under vacuum to remove approximately half the solvent. Heptane (400 mL) was added and the vacuum distillation continued until approximately 300 mL reaction product slurry remained. The product was isolated by filtration and dried under vacuum at 50° C. for 4 h. to yield 2,3-dimethyl-6-amino-2H-indazole as the free base. (40.76 g, 96.7%). ¹H NMR (300 MHz, DMSO-d₆) δ 7.31 (d, J=8.9 Hz, 1H), 6.45 (d, J=8.9 Hz, 1H), 6.38 (s, 1H), 4.95 (s, br, 2H), 3.85 (s, 3H), 2.44 (s, 3H) MS (ES+, m/z) 162 (M+H).

### Intermediate Example 3

### Preparation of N-(2-chloropyrimidin-4-yl)-2,3-dimethyl-2H-indazol-6-amine

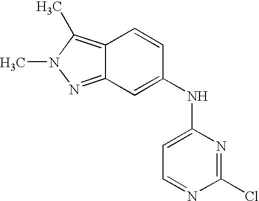 Procedure 1

To a stirred solution of the product of Intermediate Example 2 (2.97 g, 0.015 mol) and NaHCO₃ (5.05 g, 0.06 mol) in THF (15 mL) and ethanol (60 mL) was added 2,4-dichloropyrimidine (6.70 g, 0.045 mol) at rt. After the reaction was stirred for four hours at 85° C., the suspension was cooled to rt, filtered and washed thoroughly with ethyl acetate. The filtrate was concentrated under reduced pressure, and the resulting solid was triturated with ethyl acetate to yield N-(2-chloropyrimidin-4-yl)-2,3-dimethyl-2H-indazol-6-amine (89%, 3.84 g). ¹H NMR (400 MHz, DMSO-d₆) δ 7.28 (d, J=9.0 Hz, 1H), 6.42 (d, J=8.8 Hz, 1H), 6.37 (s, 1H), 5.18 (br s, 1H), 3.84 (s, 3H), 2.43 (s, 3H). MS (ES+, m/z) 274 (M+H).

Procedure 2

To a 1-L 3-necked flask equipped with air-driven mechanical stirrer, thermometer, and nitrogen inlet/outlet was charged a solution of the product of Intermediate Example 2 (32.89 g, 0.204 mol, 1.0 equiv) in 425 mL (13 volumes) of EtOH/THF (4/1), sodium bicarbonate (51.42 g, 0.612 mol, 3.0 equiv) and then 2,4-dichloropyrimidine (45.59 g, 0.306 mol. 1.5 equiv). The flask contents were heated to 75° C. and held at 74-76° C. for 6-7 hrs. The progress of the reaction was checked by HPLC (the product of Intermediate Example 2<20%). The reaction contents were cooled to 20-25° C. over 30 min, and kept at 20-25° C. for 30 min. Then the reaction contents were further cooled to 10-12° C. over 30 min, and kept at that temperature for an additional 10 min. The contents were filtered and filter cake washed with EtOAc (2×100 mL, 3.0 volumes), and deionized water (514 mL, 15.6 volumes). The filter cake was then dried in a vacuum oven at 35° C. overnight to afford the desired product 44.75 g as a white solid (80.1%). 1H NMR (400 MHz, DMSO-d₆) δ 7.28 (d, J=9.0 Hz, 1H), 6.42 (d, J=8.8 Hz, 1H), 6.37 (s, 1H), 5.18 (br s, 1H), 3.84 (s, 3H), 2.43 (s, 3H). MS (ES+, m/z) 274 (M+H).

### Intermediate Example 4

### Preparation of N-(2-chloropyrimidin-4-yl)-N,2,3-trimethyl-2H-indazol-6-amine

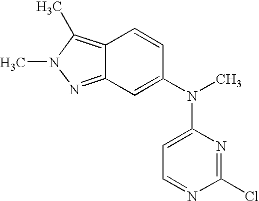 Procedure 1

To a stirred solution of the product of Intermediate Example 3 (7.37 g) in DMF (50 ml) was added Cs₂CO₃ (7.44 g, 2 eqv.) and iodomethane (1.84 ml, 1.1 eqv.) at room temperature. The mixture was stirred at rt overnight The reaction mixture was then poured into an ice-water bath, and the precipitate was collected via filtration and washed with water. The precipitate was air-dried to afford N-(2-chloropyrimidin-4-yl)-N,2,3-trimethyl-2H-indazol-6-amine as an off-white solid (6.43 g, 83%). ¹H NMR (400 MHz, DMSO-d₆) δ 7.94 (d, J=6.0 Hz, 1H), 7.80 (d, J=7.0 Hz, 1H), 7.50 (d, J=1.0 Hz, 1H), 6.88 (m, 1H), 6.24 (d, J=6.2 Hz, 1H), 4.06 (s, 3H), 3.42 (s, 3H), 2.62 (s, 3H). MS (ES+, m/z) 288 (M+H).

Procedure 2

A 3L 3-necked flask equipped with air-driven mechanical stirrer, thermometer, addition funnel and nitrogen inlet/outlet was charged with DMF (272 mL, 5 volumes) and the product of Intermediate Example 3 (54.4 g, 0.20 mol, 1.0 equiv) with stirring. The reaction mixture was further charged with cesium carbonate (194.5 g, 0.60 mol, 3.0 equiv) while maintaining the reaction temperature between 20˜25° C. The reaction mixture was stirred at 20˜25° C. for 10 minutes. lodomethane (45.1 g, 0.32 mol. 1.6 equiv) was charged over ˜10 minutes while maintaining the temperature 20˜30° C. The reaction mixture was stirred at 20˜30° C. (Typically, the reaction is complete in 1˜2 hours). Deionized H₂O (925 mL, 17 volumes) was added over ˜30 minutes while maintaining the temperature at 25˜40° C. The reaction mixture was stirred at 20˜25° C. for 40 minutes. The product was isolated by filtration and then the filter cake washed with H₂O/DMF (6:1, 252 mL, 4.6 volumes). The wet cake was dried under vacuum at 40˜45° C. and N-(2-chloropyrimidin-4yl)-N,2,3-trimethyl-2H-indazol-6-amine (51.7 g, 90.4%) was isolated as a yellow solid. ¹H NMR (400 MHz, DMSO-d₆) δ 7.94 (d, J=6.0 Hz, 1H), 7.80 (d, J=7.0 Hz, 1H), 7.50 (d, J=1.0 Hz, 1H), 6.88 (m, 1H), 6.24 (d, J=6.2 Hz, 1H), 4.06 (s, 3H), 3.42 (s, 3H), 2.62 (s, 3H). MS (ES+, m/z) 288 (M+H).

### Intermediate Example 5

### Preparation of 5-amino-2-methylbenzenesulfonamide

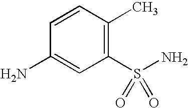 Procedure 1

To a stirred solution of 2-methyl-5-nitrobenzenesulfonamide (4.6 g, 0.021 mol) in 2-methoxyethyl ether (43 mL), at 0° C., was added a solution of 16.1 g of tin(II) chloride in 32 mL of concentrated HCl dropwise over 15 min. After the addition was complete, the ice bath was removed and the solution was allowed to stir for an additional 30 min. Approximately 130 mL of diethyl ether was added to reaction. The mixture was stirred vigorously for 1 h. The mixture was basified with a solution of NaOH and NaHCO3, and extracted with ethyl acetate (×3). The combined ethyl acetate layers were dried over anhydrous MgSO₄, filtered and concentrated to give crude product Trituation of the crude product with methanol provided 2.4 g of pure 5-amino-2-methylbenzenesulfonamide as light brown solid. ¹H NMR (300 MHz, DMSO-d₆) δ 7.11-7.10 (m, 3H), 6.95 (d, J=8.1 Hz, 1H), 6.60 (dd, J=8.1 εt 2.4 Hz, 1H), 5.24 (s, 2H), 2.36 (s, 3H). MS (ES+, m/z) 187 (M+H).

### Intermediate Example 6

### Preparation of 4-[(methylsulfonyl)methyl]aniline

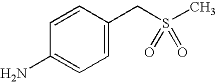 Procedure 1

Combine 4-nitrobenzyl bromide (40 g, 0.185 mol) and sodium methanesulphinic acid (19.5 g, 1 eqv.) in ethanol (460 mL, ˜0.4M). The mixture was stirred and heated to 80° C. under reflux. After 3 hr the reaction mixture was cooled to rt and filtered to collected off-white solid. The solid was washed with EtOH twice and air-dried to provide 37 g of methyl 4-nitrobenzyl sulfone. ¹H NMR (300 MHz, DMSO-d₆) δ 8.27 (d, J=8.6 Hz, 2H), 7.69 (d, J=8.6 Hz, 2H), 4.71 (s, 2H), 2.96 (s, 3H). MS (ES+, m/z) 216 (M+H).

Combined methyl 4-nitrobenzyl sulfone (9.5 g, 0.044 mol) and 10% Pd/C (0.95 g, 0.1 w/w) in ethyl acetate (220 mL ˜0.2M). The mixture was placed under Parr shaker with 40 psi of hydrogen. After ˜3 hr. the reaction mixture was poured into 50% of MeOH/EtOAc (400 mL) and stirred vigorously for 30 min. The mixture was filtered through a pad of celite and silica gel. The black material on top of the pad was removed and placed into 80% MeOH/EtOAc (200 mL) and stirred vigorously for 30 min. The mixture was again filtered through a pad of celite and silica gel. The process is repeated a couple times. Combined all filtrates. Evaporated and dried. Trituation with EtOAc provided pure 4-[(methylsulfonyl)methyl]aniline. ¹H NMR (300 MHz, DMSO-d₆) δ 7.03 (d, J=8.4 Hz, 2H), 6.54 (d, J=8.6 Hz, 2H), 5.20 (s, 2H), 4.20 (s, 2H), 2.79 (s, 3H). MS (ES+, m/z) 186 (M+H).

Procedure 2

Charge a round bottom flask (1.0 L), equipped with magnetic stir bar and reflux condenser, with 4-nitrobenzyl bromide (40 g, 0.185 mol, 1.0 eq.), sodium methanesulphinic acid (21.7 g, 0.213 mol. 1.15 eq.) and ethanol (400 mL, 200 proof, 10 vol.). Stir and heat the mixture to 80° C. under reflux for 2 hours. Check the progress of the reaction by fast-HPLC (reaction is deemed complete when HPLC indicates 4-nitrobenzyl bromide <0.5%). Cool the mixture to room temperature. Filter and wash the cake with ethanol (40 mL). The wet cake (15 g, 46.2 mmol) was used for next step hydrogenation with out further dry.

Charge a 500 mL of hydrogenation flask with above wet cake methyl 4-nitrobenzyl sulfone (15 g, 46.2 mmol, used “as is”), 10% Pd/C (0.1 g, 1% wiw) and ethanol (120 mL, 200 proof) and water (40 mL). Swap the atmosphere. of reactor with hydrogen (3 times). Shake the reactor under H₂ (65 psi) at room temperature for 30 minutes and at 50° C. for two hour. Check the progress of the reaction by HPLC (reaction is deemed complete when HPLC indicates methyl 4-nitrobenzyl sulfone <0.2%). Heat the mixture to 80° C. Filter the hot solution through a pad of celite (2.0 g) and rinse the pad with EtOH (10 mL). Transfer the filtrate into the crystallizing a round bottom flask (500 mL). Distil the slurry under house vacuum at 60° C. until a volume of 60 mL is left Cool the slurry to 0° C. over for one hour. Isolate the crystals by vacuum filtration and wash the vessel and crystals with ethanol (10 mL). Dry the product under house vacuum at 50° C. to constant weight. Obtained off-white solid (7.3 g). The yield is 85% for combined two steps with 99% purity of product by HPLC.

### Intermediate Example 7

### Preparation of 4-[(isopropylsulfonyl)methyl]phenylamine

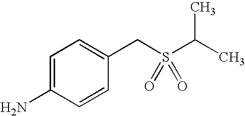

To a solution of 1-(bromomethyl)-4-nitrobenzene (3.0 g, 17.4 mmol) in ethanol (50 mL) was added sodium-2-thiopropoylate (2.7 g, 17.4 mmol). After 12 h the solvent was removed under reduced pressure, the remaining residue was diluted with EtOAc and filtered to remove the residual salts. The solvent was dried over MgSO₄ and removed under reduced pressure and the product was carried forward without further purification. Next the sulfide was diluted with CH₂Cl₂ (50 mL) and m-chloroperoxybenzoic acid (˜700/%) (6.6 g, 38.4 mmol) was added in portions. The reaction was judged to be complete by tic and the solvent was removed under reduced pressure: The remaining residue was diluted with EtOAc and washed with 1M NaOH (2×100 mL). The solvent was dried over MgSO₄ and removed under reduced pressure and the product was carried forward without further purification. Next the residue was diluted with glyme (8.0 mL) and a solution of SnCl₂ (13.8 g, 69 mmol) in HCl (8.0 mL) was added dropwise. The solution was allowed to stir for 2 h, and the reduction was judged to be complete by tlc. The reaction mixture was diluted with Et₂O, which resulted in the precipitation of the product as the HCl salt. The solids were collected and washed with Et₂O (2×100 mL), to afford pure aniline (˜2.4 g, 65%). ¹H NMR (300 MHz, d₆DMSO+NaHCO₃) δ 7.37 (d, J=8.4 Hz, 2H), 7.21 (d, J=8.4 Hz, 2H), 4.41 (s, 2H), 3.18-3.09 (m, 1H), 1.21 (d, J=6.9 Hz, 6H).

### Intermediate Example 8

### Preparation of 4-[2-(methylsulfonyl)ethyl]aniline

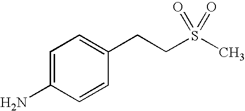

To a solution of 1-(bromoethyl)-4-nitrobenzene (3.0 g, 13.0 mmol) in ethanol (70 mL) was added Sodium thiomethoxide (1.0 g, 14.0 mmol). After 12 h the solvent was removed under reduced pressure, the remaining residue was diluted with EtOAc and filtered to remove the residual salts. The solvent was dried over MgSO4 and removed under reduced pressure and the product was carried forward without further purification. Next the sulfide was diluted with CH₂Cl₂ (100 mL) and m-chloroperoxybenzoic acid (˜70%) (8.2 g, 48.8 mmol) was added in portions. The reaction was judged to be complete by tic and the solvent was removed under reduced pressure. The remaining residue was diluted with EtOAc and washed with 1M NaOH (2×100 mL). The solvent was dried over MgSO₄ and removed under reduced pressure and the product was carried forward without further purification. Next the residue was added to a slurry of Palladium on Carbon (10 mol %) in EtOAc (50 mL) in a Parr shaker vessel. The reaction was then place under 40 atm of Hydrogen gas. The solution was allowed to shake for 2 h, and the reduction was judged to be complete by tic. The reaction mixture was filtered over a pad of celite and washed with EtOAc and the solvent was removed under reduced pressure to afford a crude solid. The mixture was recrystallized in hot EtOAc to afford the pure aniline (˜1.8 g, 69%). ¹H NMR (300 MHz, d₆DMSO+NaHCO₃) δ 6.93 (d, J=8.2 Hz, 2H), 6.87 (d, J=8.2 Hz, 2H), 5.09 (bs, 2H), 3.31-3.26 (m, 2H), 2.92 (s, 3H), 2.84-2.79 (m, 2H).

### Intermediate Example 9

### Preparation of 4-[1-(methylsulfonyl)ethyl]aniline

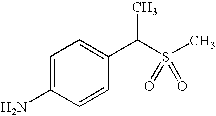

To a solution of 4-nitrophenylcarbonol (3.0 g, 17.9 mmol) and triethylamine (3.5 mL, 21.0 mmol) in CH₂Cl₂ (100 mL) was added methanesulfonylchloride (1.7 mL, 21.0 mmol) dropwise. The reaction was judged to be complete by tic after 1 h and was quenched with saturated aqueous NaHCO3. The reaction mixture was diluted with EtOAc and the organic layer separated, dried over MgSO₄ and the solvent was removed under reduced pressure. The resulting residue was dissolved in ethanol (100 mL) and Sodium thiomethoxide (1.5 g, 21.0 mmol) was added in portions. After 12 h the solvent was removed under reduced pressure, the remaining residue was diluted with EtOAc and filtered to remove the residual salts. The solvent was dried over MgSO₄ and removed under reduced pressure and the product was carried forward without further purification. Next the sulfide was diluted with CH₂Cl₂ (100 mL) and m-chloroperoxybenzoic acid (˜70%) (10.8 g, 62 mmol) was added in portions. The reaction was judged to be complete by tic and the solvent was removed under reduced pressure. The remaining residue was diluted with EtOAc and washed with 1 M NaOH (2×100 mL). The solvent was dried over MgSO₄ and removed under reduced pressure and the product was carried forward without further purification. Next the residue was added to a slurry of Palladium on Carbon (10 mol %) in EtOAc (50 mL) in a Parr shaker vessel. The reaction was then place under 40 atm of Hydrogen gas. The solution was allowed to shake for 2 h, and the reduction was judged to be complete by tlc. The reaction mixture was filtered over a pad of celite and washed with EtOAc and the solvent was removed under reduced pressure to afford a crude solid. The mixture was recrystallized in hot EtOAc to afford the pure aniline (˜2.0 g, 57%). ¹H NMR (300 MHz, d₆DMSO+NaHCO₃) δ 7.06 (d, J=8.5 Hz, 2H), 6.53 (d, J=8.5 Hz. 2H), 5.21 (s, 2H), 4.23 (q, J=7.1 Hz, 1H), 2.70 (s, 3H), 1.21 (d, J=7.1 Hz, 3H).

### Intermediate Example 10

### Preparation of 4-[1-methyl-1-(methylsulfonyl)ethyl]aniline

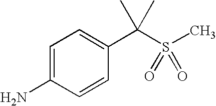

To a stirred solution of t-butoxide (5.76 g, 0.051 mol) in THF was added methyl 4-nitrobenzyl sulfone (5 g, 0.023 mol) followed by iodomethane (2.89 ml, 0.046 mol). The mixture was stirred at rt for 1 hr. Additional t-butoxide (2.9 g) and iodomethane (0.5 ml) were added. The mixture was stirred at rt for additional 1 hr. The mixture was diluted with EtOAc and acidified with 6N HCl. The mixture was extracted with ethyl acetate (×3). The combined ethyl acetate layers were dried over anhydrous MgSO4, filtered and evaporated. The solid was trituated with ethanol to give pure 1-[1-methyl-1-(methylsu lfonyl)ethyl]-4-nitrobenzene.

To a stirred solution of 1-[1-methyl-1-(methylsulfonyl)ethyl]-4-nitrobenzene (3.32 g, 0.014 mol) in 2-methoxyethyl ether (70 mL), at 0° C., was added a solution of 10.35 g of tin(II) chloride in 20.5 mL of concentrated HCl dropwise over 15 min. After the addition was complete, the ice bath was removed and the solution was allowed to stir for an additional 30 min. Approximately 70 mL of diethyl ether was added to reaction. The mixture was stirred vigorously for 1 h. Precipitate was formed and was collected via filtration. The solid was dissolved in CH₂Cl₂ and washed with 1N NaOH. The mixture was extracted with CH₂Cl₂ (×3). The combined CH₂Cl₂ layers were dried over anhydrous MgSO₄, filtered and evaporated to give 4-[1-methyl-1-(methylsulfonyl)ethyl]aniline as an off white solid. ¹H NMR (300 MHz, DMSO-d₆) δ 7.21 (d, J=8.6 Hz, 2H), 6.55 (d, J=8.6 Hz, 2H), 5.23 (s, 2H), 2.58 (s, 3H), 1.64 (S, 6H).

### Example 1

### 5-({4-[(2,3-dimethyl-2H-indazol-6-yl)(methyl)amino]pyrimidin-2-yl}amino)-2-methylbenzenesulfonamide

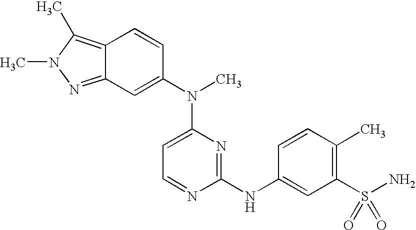 Procedure 1

To a solution of Intermediate Example 4 (200 mg, 0.695 mmol) and 5-amino-2-methylbenzenesulfonamide (129.4 mg, 0.695 mmol) in isopropanol (6 ml) was added 4 drops of conc. HCl. The mixture was heated to reflux overnight The mixture was cooled to rt and diluted with ether (6 ml). Precipitate was collected via filtration and washed with ether. The hydrochloride salt of 5-({4-[(2,3-dimethyl-2H-indazol-6-yl)(methyl)amino]-pyrimidin-2-yl}amino)-2-methylbenzenesulfonamide was isolated as an off-white solid. ¹H NMR (400 MHz, d₆DMSO+NaHCO₃) δ 9.50 (br s, 1 H), 8.55 (br s, 1H), 7.81 (d, J=6.2 Hz, 1H), 7.75 (d, J=8.7 Hz, 1H), 7.69 (m, 1H), 7.43 (s, 1H), 7.23 (s, 2H), 7.15 (d, J=8.4 Hz, 1H), 6.86 (m, 1H), 5.74 (d, J=6.1 Hz, 1H), 4.04 (s, 3H), 3.48 (s, 3H), 2.61 (s, 3H), 2.48 (s, 3H). MS (ES+, m/z) 438 (M+H).

Procedure 2

A 250-mL 3-necked flask equipped with a magnetic stir bar, thermometer, reflux condenser, and nitrogen inlet/outlet was charged with ethanol (60 mL, 10 volumes), the product of Intermediate Example 4 (6.00 g, 20.85 mmol, 1.0 equiv) and 5-amino-2-methylbenzenesulfonamide (4.00 g, 21.48 mmol, 1.03 equiv) with stirring. The reaction mixture was heated to 70° C. After stirring the reaction mixture at 68-72° C. for 3 hrs, 4M HCl in dioxane (0.11 mL 0.44 mmol, 0.02 equiv) was charged over ca. 2 min. The reaction mixture was stirred at 68-72° C. until <1.5% by area of the starting product of Intermediate Example 4 was remaining by HPLC analysis (Typically, this reaction is complete in >8 hrs). The reaction mixture was cooled to 20° C. over ca. 30 min and stirred at 20-22° C. for 40 min. The product was then isolated by filtration and the filter cake washed with ethanol (20 mL 3.3 volumes). The wet cake was dried under vacuum at 45-50° C. The monohydrochloride salt of 5-({4-[(2,3-dimethyl-2H-indazol-6-yl)(methyl)amino]-pyrimidin-2-yl}amino)-2-methylbenzenesulfonamide (9.52 g, 96.4%) was isolated as a white solid. ¹H NMR (400 MHz, d₆DMSO+NaHCO₃) δ 9.50 (br s, 1H), 8.55 (br s, 1H), 7.81 (d, J=6.2 Hz, 1H), 7.75 (d, J=8.7 Hz, 1H), 7.69 (m, 1H), 7.43 (s, 1H), 7.23 (s, 2H), 7.15 (d, J=8.4 Hz, 1H), 6.86 (m, 1H), 5.74 (d, J=6.1 Hz, 1H), 4.04 (s, 3H), 3.48 (s, 3H), 2.61 (s, 3H), 2.48 (s, 3H), MS (ES+, m/z) 438 (M+H).

Procedure 3:

To a stirred suspension of the product of Intermediate Example 4 (1.1 g, 3.8 mmol) in 14 mL of MeOH, was added 5-amino-2-methylbenzenesulfonamide (0.78 g, 4.2 mmol, 1.1 equiv) at room temperature. The reaction mixture was heated at reflux for 3 h, then 4 M HCl in 1,4-dioxane (19 μL, 0.076 mmol) was added in one portion. After 4 h, the suspension was cooled to room temperature, and filtered. The resulting solid was washed with 10 mL of MeOH and dried in vacuo to yield 1.3 g (72%) of 5-({4-[(2,3-dimethyl-2H-indazol-6-yl)methylamino]-2-pyrimidinyl}amino)-2-methyl benzenesulfonamide monohydrochloride as a white solid. ¹H NMR (DMSO-d6, 400 MHz) δ 10.95 (s, 1H), 8.36 (s, 1H), 7.86 (d, J=8.8 Hz, 2H), 7.64-7.59 (m, 2H), 7.40 (m, 3H), 6.93 (dd, J=8.8, 2.0 Hz, 1H), 5.92 (s, 1H), 4.08 (s, 3H), 3.57 (s, 3H), 2.65 (s, 3H), 2.56 (s, 3H).

Procedure 4

To a stirred suspension of the product of Intermediate Example 4 (1.1 g, 3.7 mmol) in 10 mL of THF, was added 5-amino-2-methylbenzenesulfonamide (0.70 g, 3.8 mmol, 1.0 equiv) at room temperature. The reaction mixture was heated at reflux for 3 h, then 4 M HCl in 1,4-dioxane (18 μL 0.072 mmol) was added in one portion. After 5 h, the suspension was cooled to room temperature, and filtered. The resulting solid was washed with 16 mL of THF and dried in the air to yield 1.6 g (92%) of 5-({4-[(2,3-dimethyl-2H-indazol-6-yl)methylaminol-2-pyrimidinyl}amino)-2-methylbenzene sulfonamide monohydrochloride as a light yellow solid.

Procedure 5

To a stirred suspension of the product of Intermediate Example 4 (1.0 g, 3.6 mmol) in 10 mL of CH₃CN, was added 5-amino-2-methylbenzenesulfonamide (0.70 g, 3.8 mmol, 1.0equiv) at room temperature. The reaction mixture was heated at reflux for 3 h, then 4 M HCl in 1,4-dioxane (18 μL, 0.076 mmol) was added in one portion. After 20 h, the suspension was cooled to room temperature, and filtered. The resulting solid was washed with 10 mL of CH₃CN and dried in the air to yield 1.3 g (73%) of 5-({4-[(2,3-dimethyl-2H-indazol-6-yl)methylamino]-2-pyrimidinyllamino)-2-methyl benzenesulfonamide monohydrochloride as an off-white solid.

Procedure 6

### Preparation of 5-({4-[(2,3-dimethyl-2H-indazol-6-yl)(methyl)amino]pyrimidin-2-yl}amino)-2-methylbenzenesulfonamide methanesulfonic acid salt

In a 250 mL flask the product of Example 1, procedure 1, (1.0 g, 2.29 mmol) was slurried in water (19 mL). Methanesulfonic acid (0.231 g, 2.4 mmol) was added all at once and the mixture was heated to reflux for 5 min. The mixture was cooled to 0° C. over a 1 hour period and was then isolated by filtration and air dried. 5-({4-[(2,3-Dimethyl-2H-indazol-6-yl)(methyl)amino]pyrimidin-2-yl}amino)-2-methylbenzenesulfonamide methanesulfonic acid salt (1.03 g, 84%) was obtained as a white solid. mp=247-248° C.

Procedure 7:

### Preparation of 5-({4-[(2,3-dimethyl-2H-indazol-6-yl)methylamino]-2-pyrimidinyl}amino)-2-methylbenzenesulfonamide monohydrochloride monohydrate

To a round bottom flask, was added 2.6 g of the monohydrochloride salt of Example 1, procedure 1, any form. Then added was 39 mL of isopropanol (15 volumes). The mixture was heated to 75 deg C in an oil bath, then 14 mL of 0.05N aqueous HCl (5.4 volumes) was added. The clear solution was cooled to 65 deg C, then seeded with the monohydrate of the monohydrochloride salt of Example 1, procedure 1 (0.05-0.1 wt %). The cloudy solution was stirred at 65 deg C for 60 minutes, then cooled to 0 deg C at ˜0.25-0.5 deg C/min. The resulting white solid was filtered and dried to constant weight under vacuum at RT to give 88% yield of 5-({4-[(2,3-dimethyl-2H-indazol-6-yl)methylamino]-2-pyrimidinyl}amino)-2-methylbenzene sulfonamide monohydrochloride monohydrate.

The following examples were prepared according to the general procedure set forth above in Example 1 using Intermediate Example 4 and the appropriate aniline. The appropriate anilines were prepared using procedures similarly described for Intermediate Examples 5-10.

### Example 2

### N⁴-(2,3-dimethyl-2H-indazol-6-yl)-N⁴-methyl-N²-{4-[(methylsulfonyl)methyl]phenyl}pyrimidine-2,4-diamine

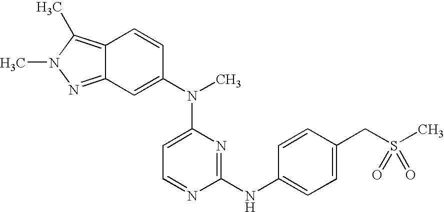

¹H NMR (300 MHz, Na₂CO₃+DMSO-d₆) δ 9.37 (bs, 1H). 7.88 (d, J=6.1 Hz, 1H), 7.78 (m, 3H), 7.47 (s, 1H), 7.22 (d, J=8.5 Hz, 2H), 6.91 (dd, J=8.8, 1.5 Hz, 1H), 5.84 (d, J=6.1 Hz, 1H), 4.37 (s, 2H), 4.09 (s, 3H), 3.51 (s, 3H), 2.88 (s, 3H), 2.65 (s, 3H). MS (ES+, m/z) 437 (M+H), 435 (M−H).

### Example 3

### 3-({4-[(2,3-dimethyl-2H-indazol-6-yl)(methyl)amino]pyrimidin-2-yl}amino)benzenesulfonamide

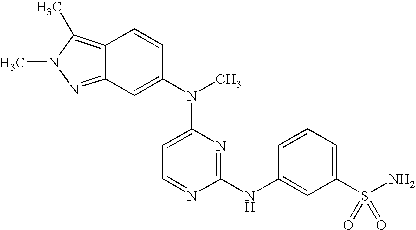

¹H NMR (400 MHz, DMSO-d₆+NaHCO₃) δ 9.58 (br s, 1H), 8.55 (br s, 1H), 7.83 (d, J=6.2 Hz, 1H), 7.74-7.79 (m, 2H), 7.43 (s, 1H), 7.34-7.37 (m, 2H), 7.24 (s, 2H), 6.86 (m, 1H), 5.77 (d, J=6.1 Hz, 1H), 4.04 (s, 3H), 3.48 (s, 3H), 2.61 (s, 3H). MS (ES+, m/z) 424 (M+H).

### Example 4

### 2-[4-({4-[(2,3-dimethyl-2H-indazol-6-yl)(methyl)amino]pyrimidin-2-yl}amino)phenyl]ethanesulfonamide

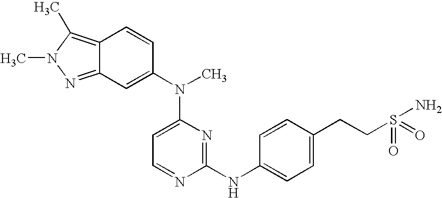

¹H NMR (300 MHz, Na₂CO₃+DMSO-d₆) δ 9.10 (br s, 1H), 7.83 (d, J=6.0 Hz, 1H), 7.75 (d, J=8.7 Hz, 1H), 7.67 (d, J=8.5 Hz, 2H), 7.43 (d, J=1.1 Hz, 1H), 7.06 (d, J=8.5 Hz, 2H), 6.86-6.89 (m, 3H), 5.76 (d, J=6.0 Hz, 1H), 4.06 (s, 3H), 3.46 (s, 3H), 3.21 (m, 2H), 2.91 (m, 2H), 2.62 (s, 3H). MS (ES+, m/z) 452 (M+H).

### Example 5

### 2-[4-({4-[(2,3-dimethyl-2H-indazol-6-yl)(methyl)amino]-2-pyrimidinyl}amino)phenyl]-N-methylethanesulfonamide

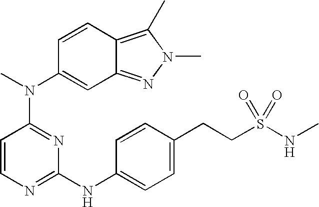

¹H NMR (300 MHz, Na₂CO₃+DMSO-d₆) δ 9.09 (s, 1H), 7.82 (d, J=6.0 Hz, 1H), 7.75 (d, J=8.8 Hz, 1H), 7.67 (d, J=8.5 Hz, 2H), 7.43 (d, J=1.0 Hz, 1H), 7.08 (d, J=8.5 Hz, 2H), 6.94 (q, J=5.0 Hz, 1H), 6.87 (dd, J=8.8 εt 1.6 Hz, 1H), 5.76 (d, J=6.0 Hz, 1H), 4.05 (s, 3H), 3.46 (s, 3H), 3.22 (m, 2H), 2.84 (m, 2H), 2.62 (s, 3H), 2.59 (d, J=5.0 Hz, 3H). MS (ESI) m/z=466 [M+H]⁺.

### Example 6

### 2-[3-({4-[(2,3-dimethyl-2H-indazol-6-yl)(methyi)amino]-2-pyrimidinyl}amino)phenyl]ethanesulfonamide

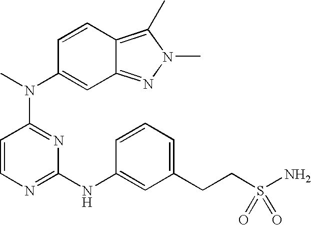

¹H NMR (300 MHz; Na₂CO₃+DMSO-d₆) δ 9.13 (s, 1H), 7.84 (d, J=5.9 Hz, 1H), 7.77-7.72 (m, 2H), 7.58 (d, J=8.2 Hz, 1H), 7.44 (s, 1H), 7.12 (m, 1H), 6.89-6.86 (m, 3H), 6.77 (d, J=7.5 Hz, 1H), 5.77 (d, J=6.0 Hz, 1H), 4.05 (s, 3H), 3.47 (s, 3H), 3.20 (m, 2H), 2.92 (m, 2H), 2.62 (s, 3H). MS (ESI) m/z=452 [M+H]⁺.

### Example 7

### 2-chloro-5-({4-[(2,3-dimethyl-2H-indazol-6-yl)(methyl)amino]-2-pyrimidinyl}amino)benzenesulfonamide

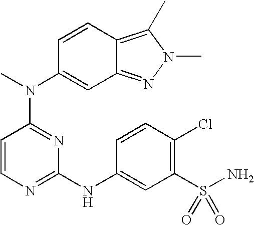

¹H NMR (300 MHz, Na₂CO₃+DMSO-d₆) δ 9.63 (s, 1H), 8.76 (s, 1H), 7.86-7.82 (m, 2H), 7.77 (d, J=8.8 Hz, 1H), 7.46-7.45 (m, 3H), 7.39 (d, J=8.8 Hz, 1H), 6.88 (d, J=8.8 Hz, 1H), 5.79 (d, J=6.0 Hz, 1H), 4.06 (s, 3H), 3.49 (s, 3H), 2.62 (s, 3H). MS (ESI m/z=458 [M+H]⁺.

### Example 8

### N⁴-(2,3-dimethyl-2H-indazol-6-yl)-N⁴-methyl-N²-{3-[1-(methylsulfonyl)ethyl]phenyl}-2,4-pyrimidinediamine

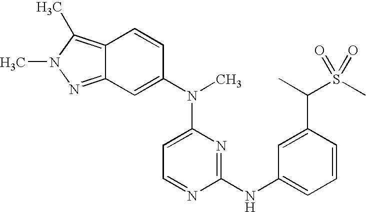

¹H NMR (300 MHz, d₆-DMSO) δ 9.24 (s, 1H), 7.92 (3, 1H), 7.86 (d, J=5.8 Hz, 1H), 7.75 (d, J=9.7 Hz, 1H), 7.62 (d, J=8.0 Hz, 1H), 7.44 (s, 1H), 7.19 (dd, J=7.9 and 7.6 Hz, 1H), 6.95 (d, J=7.6 Hz, 1H), 6.88 (d, J=8.4 Hz, 1H), 5.82 (d, J=5.9 Hz, 1H), 4.17 (q, J=7.0 Hz, 1H), 4.05 (s, 3H), 3.47 (s, 3H), 2.75 (s, 3H), 2.62 (s, 3H), 1.57 (d, J=5.7 Hz, 3H) ppm. MS (ESI) m/z=451 [M+H]⁺.

### Example 9

### N⁴-(2,3-dimethyl-2H-indazol-6-yl)-N⁴-methyl-N²-{4-[1-(methylsulfonyl)ethyl]phenyl}-2,4-pyrimidinediamine

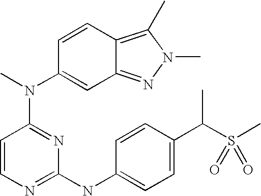

¹H NMR (300 MHz, d₆-DMSO) δ 9.25 (s, 1H), 7.86 (d, J=5.8, 1H), 7.75 (d, J=8.5 Hz, 1H), 7.73 (d, J=6.9 Hz, 2H), 7.44 (s, 1H), 7.22 (d, J=8.5 Hz, 2H), 6.88 (d, J=8.8 Hz, 1H), 5.81 (d, J=5.8 Hz, 1H), 4.39 (q, J=7.2, Hz, 1H), 4.06 (s, 3H), 3.47 (m, 3H), 2.76 (s, 3H), 2.63 (s, 3H), 1.58 (d, J=7.2 Hz, 3H) ppm. MS (ESI) m/z=451 [M+H]⁺.

### Example 10

### N⁴-(2,3-dimethyl-2H-indazol-6-yl)-N⁴-methyl-N²-{4-[1-methyl-1-(methylsulfonyl)ethyl]phenyl}-2,4-pyrimidinediamine

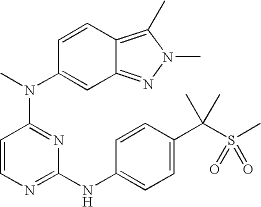

¹H NMR (300 MHz, Na₂CO₃+DMSO-d₆) δ 9.26 (s, 1H), 7.86 (d, J=5.8 Hz, 1H), 7.77-7.72 (m, 3H), 7.44 (s, 1H), 7.36 (d, J=8.8 Hz, 2H), 6.88 (dd, J=8.8 εt 1.5 Hz, 1H), 5.83 (d, J=6.0 Hz, 1H), 4.06 (s, 3H), 3.47 (s, 3H), 2.63 (s, 6H), 1.69 (s, 6H). MS m/z=465 [M+H]⁺.

### Example 11

### N⁴-(2,3-dimethyl-2H-indazol-6-yl)-N⁴-methyl-N²-(4-[(4-methylphenyl)sulfonyl]methyl}phenyl)-2,4-pyrimidinediamine

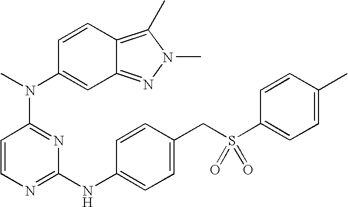

¹H NMR (300 MHz, Na₂CO₃+DMSO-d₆) δ 9.19 (s, 1H), 7.84 (d, J=6.0 Hz, 1H), 7.74 (d, J=8.7 Hz, 1H), 7.63 (d, J=8.5 Hz, 2H), 7.57 (d, J=8.2 Hz, 2H), 7.43-7.37 (m, 3H), 6.93-6.86 (m, 3H), 5.79 (d, J=6.0 Hz, 1H), 4.48 (s, 2H), 4.06 (s, 3H), 3.45 (s, 3H), 2.63 (s, 3H), 2.39 (s, 3H). MS (ESI) m/z=513 [M+H]⁺.

### Example 12

### N⁴-(2,3-dimethyl-2H-indazol-6-yl)-N⁴-methyl-N²-(4-{ [(4-methylphenyl)sulfonyl]methyl}phenyl)-2,4-pyrimidinediamine

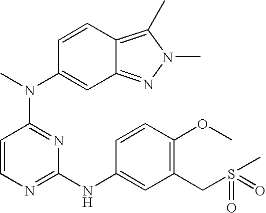

¹H NMR (300 MHz, Na₂CO₃+DMSO-d₆) δ 9.04 (s, 1H), 7.81 (d, J=5.8 Hz, 1H), 7.76-7.73 (m, 2H), 7.64 (dd, J=9.0 εt 2.7 Hz, 1H), 7.42 (s, 1H), 6.92 (d, J=9.0 Hz, 1H), 6.87 (dd, J=8.8 εt 1.6 Hz, 1H), 5.76 (d, J=5.9 Hz, 1H), 4.23 (s, 2H), 4.05 (s, 3H), 3.76 (s, 3H), 3.45 (s, 3H), 2.84 (s, 3H), 2.62 (s, 3H). MS (ESI) m/z=467 [M+H]⁺.

### Example 13

### N⁴-(2,3-dimethyl-2H-indazol-6-yl)-N⁴-methyl-N²-(4-{[(1-methylethyl)sulfonyl]methyl}phenyl)-2,4-pyrimidinediamine

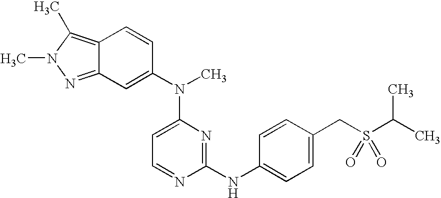

¹H NMR (300 MHz, d₆DMSO+TFA) δ 10.7 (bs, 1H), 7.86 (d, J=8.7 Hz, 1H), 7.60 (m, 3H), 7.49 (d, J=8.5 Hz, 2H), 7.35 (d, J=8.4 Hz, 2H), 6.94 (dd, J=8.8 εt 1.8 Hz, 1H), 4.50 (s, 1H), 4.43 (bs, 1H), 4.08 (s, 3H), 3.56 (s, 3H), 3.20 (m, 1H), 2.65 (s, 3H), 1.27 (m, 6H). MS (ES+, m/z) 465 (M+H).

### Example 14

### 1-[3-({4-[(2,3-dimethyl-2H-indazol-6-yl)(methyl)amino]-2-pyrimidinyl}amino)phenyl]methanesuIfonamide

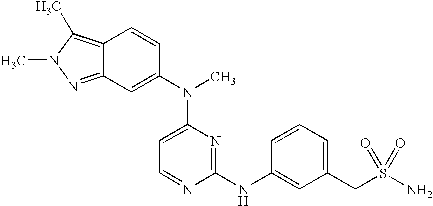

¹H NMR (300 MHz, d₆DMSO+TFA) δ 10.64 (bs, 1H), 7.87 (d, J=8.6 Hz, 1H), 7.60 (bs, 1H), 7.50 (t, J=7.6 Hz, 1H), 7.36 (m, 3H), 7.19 (bs, 1H), 6.94 (dd, J=8.8 εt 1.6 Hz, 1H), 6.90 (bs, 1H), 4.34 (s, 1H), 4.30 (bs, 1H), 4.08 (s, 3H), 3.56 (s, 3H), 2.65 (s, 3H). MS (ES+, m/z) 438 (M+H).

### Example 15

### 1-[4-({4-[(2,3-dimethyl-2H-indazol-6-yl)(methyl)amino]-2-pyrimidinyl}amino)phenyl]methanesulfonamide

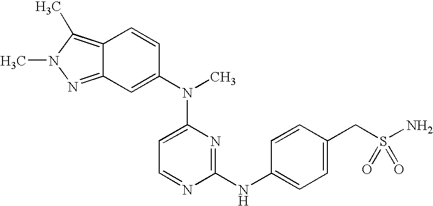

¹H NMR (300 MHz, d₆DMSO+TFA) δ 10.63 (bs, 1H), 7.85 (m, 2H), 7.60 (m, 2H), 7.46 (d, J=8.4 Hz. 2H), 7.34 (d, J=8.4 Hz, 2H), 6.94 (dd, J=8.8 εt 1.8 Hz, 1H), 6.86 (bs, 1H), 4.30 (s, 1H), 4.25 (bs, 1H), 4.08 (s, 3H), 3.56 (s, 3H), 2.65 (s, 3H). MS (ES+, m/z) 438 (M+H).

### Example 16

### N4-(2,3-dimethyl-2H-indazol-6-yl)-N4-methyl-N2-{4-[2-(methylsulfonyl)ethyl]phenyl}pyrimidine-2,4-diamine

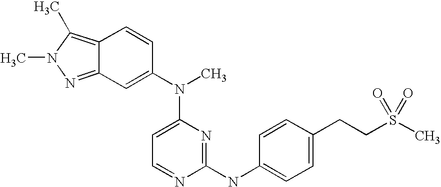

¹H NMR (300 MHz, d₆DMSO+NaHCO₃) δ 9.11 (s, 1H), 7.83 (d, J=6.1 Hz, 1H), 7.76 (d, J=7.2 Hz, 1H), 7.68 (d, J=8.5 Hz, 2H), 7.43 (s, 1H), 7.10 (d, J=8.5 Hz, 2H), 6.88 (dd, J=8.7 εt1.5 Hz, 1H), 5.76 (d, J=6.0 Hz, 1H), 4.06 (s, 3H), 3.46 (s, 3H), 3.41-3.26 (m, 2H), 2.95 (s, 3H), 2.94-2.89 (m, 2H), 2.63 (s, 3H). MS (ES+, m/z) 450.9 (M+H).

### Example 17

### N4-(2,3-dimethyl-2H-indazol-6-yl)-N4-methyl-N2-{3-[2-(methylsulfonyl)ethyl]phenyl}pyrimidine-2,4-diamine

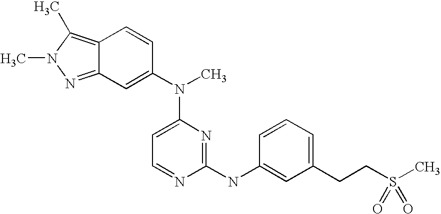

¹H NMR (300 MHz, d₆DMSO+NaHCO₃) δ 9.54 (bs, 1H), 7.84 (d, J=6.5 Hz, 1H), 7.79 (d, J=8.6 Hz, 1H), 7.53 (bs, 1H), 7.49 (bs, 2H), 7.19 (bs, 1H), 6.90 (d, J=7.6 Hz, 2H), 5.76 (d, J=6.0 Hz, 1H), 4.06 (s, 3H), 3.46 (s, 3H), 3.41-3.26 (m, 2H), 2.95 (s, 3H), 2.94-2.89 (m, 2H), 2.63 (s, 3H). MS (ES+, m/z) 451 (M+H).

### Biological Data

The compounds of the present invention elicit important and measurable pharmacological responses. Each of the compounds described in the Examples section bind with high affinity (IC₅₀<1 μM) to the kinase domain of VEGFR2 receptor, as described by the VEGFR2 HTRF assay below. In addition to binding to the kinase domain of VEGFR2, the exemplified compounds of the present invention also measurably and significantly inhibit the proliferation of endothelial cells that are stimulated for growth by activation with VEGF. Data for inhibition of cell proliferation are provided in Table 1 below.

VEGFR2 HTRF Assay

The assays were performed in 96-well black plates. 10 nM hVEGFR2 was used to phosphorylate 0.36 μM peptide (Biotin-Ahx-EEEEYFELVAKKKK) in the presence of 75 μM ATP, 5 mM MgCl₂, 0.3 mM DTT, 0.1 mg/ml BSA, and 0.1 M HEPES (pH 7.5). 10 μl 0.5 M EDTA was added to reactions as negative controls. The 50 μl kinase reaction with or without inhibitors in 5% DMSO was carried out at room temperature for 45 minutes, then stopped by 40 μl of 125 mM EDTA. 2.4 μg/ml Streptavidin-APC and 0.15 μg/ml Eu-α-pY, in the presence of 0.1 mg/ml BSA, 0.1 M HEPES (pH7.5), were added to a final volume of 140 μl. The plate was incubated for 10 min at room temperature (22° C.) and read on the Victor with the time resolved fluorescence mode by exciting at 340 nm and reading the emission at 665 nm.

Reagent Resources:

Peptide from Synpep (Dublin, Calif.)

ATP, MgCl₂, DTT, BSA, HEPES, EDTA, DMSO from Sigma

Streptavidin-APC from Molecular Probes (Eugene, Oreg.)

Eu-α-pY from EGELG Wallac (Gaithersburg, Md.)

Abbreviations:

| | |
|---|---|
| ATP | Adenosine Triphosphate |
| Streptavidin-APC | Streptavidin, allophycocyanine, crosslinked conjugate |
| DMSO | Dimethyl Sulfoxide |
| DTT | Dithiothreitol |
| BSA | Bovine Serum Albumin |
| HTRF | Homogenous Time Resolved Fluorescence |
| EDTA | Ethylenedinitrilo Tetraacetic Acid |
| HEPES | N-2-Hydroxyethyl Piperazine N-Ethane Sulfonic Acid |
| Eu-α-pY | Europium labeled anti-phosphotyrosine antibody |

Human Umbilical Vein Endothelial Cell (HUVEC) Proliferation Assay (BrdU Incorporation)
Materials

HUVEC cells and EGM-MV (Endothelial cell growth medium—microvascular) were purchased from Clonetics (San Diego, Calif.). VEGF and bFGF were purchased from RETD Systems (Minneapolis, Minn.). Anti-BrdU antibody was obtained from Chemicon International (Temecula, Calif.).

Methods

HUVECs were routinely maintained in EGM-MV medium and were used within passage 7. HUVECs were plated at a density of 2500 cellsiwell in M199 medium containing 5% FBS (Hyclone) in type I collagen coated plate (Becton Dickinson). The plate was incubated at 37° C. overnight. The medium was removed by aspiration, and test compounds were added to each well in a volume of 0.1 ml/well in serum-free M199 medium. Compound concentrations ranged from 1.5 nM to 30 micromolar. The plate was incubated for 30 min at 37° C. Another 0.1 ml of serum-free M199 medium containing BSA and VEGF (or bFGF) was added to give a final concentration of 0.10/0 BSA and 10 ng/ml VEGF (0.3 ng/ml bFGF). The plate was incubated at 37° C. for 72 hrs. BrdU was added to each well after the first 48 hrs to give a concentration of 10 micromolar. The colorimetric ELISA assay was performed according to manufacturer's (Roche Molecular Sciences) instructions, with detection by absorbance reading at 450 nm. Results were plotted as concentration of test compound vs. absorbance to give an IC₅₀ value for inhibition of BrdU incorporation.

TABLE 1

| Example No. | IC₅₀ |
|---|---|
| 1-17 | ++++ |

The application of which this description and claim.(s) forms part may be used as a basis for priority in respect of any subsequent application. The claims of such subsequent application may be directed to any feature or combination of features described herein. They may take the form of product, composition, process or use claims and may include, by way of example and without limitation, one or more of the following claim(s):

What is claimed is:

1. A process for preparing a compound of formula (R), 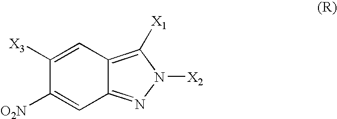
comprising the step of:
reacting a compound of formula (Q) 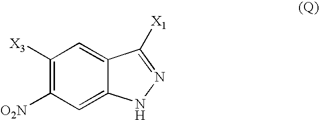
with an alkylating agent,
wherein
X₁ is hydrogen, C₁-C₄ alkyl, C₁-C₄ haloalkyl, or C₁-C₄ hydroxyalkyl;
X₂ is C₁-C₄ alkyl, C₁-C₄ haloalkyl, or aralkyl; and
X₃ is hydrogen or halogen.

2. A process for preparing a compound of formula (I)
comprising the step of:
reacting a compound of formula (Q′) 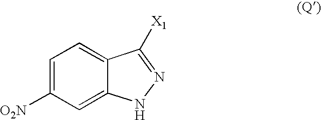
with an alkylating agent to prepare a compound of formula (R′), 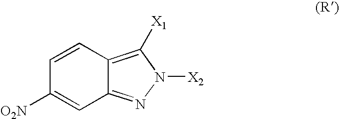
wherein:
X₁ is hydrogen or C₁-C₄ alkyl;
X₂ is C₁-C₄ alkyl or benzyl;
X₄ is hydrogen or C₁-C₄ alkyl;
Q₁ is A¹ or A²;
Q₂ is A¹ when Q₁ is A² and Q₂ is A² when Q₁ is A¹;
wherein
A¹ is hydrogen, halogen, C₁-C₃alkyl, C₁-C₃haloalkyl, C₁-C₄alkoxy, and
A² is the group defined by -(Z)ₘ-(Z¹)-(Z²), wherein
Z is C(R′)(R″), where R′ and R″ are independently selected from —H or C₁-C₄ alkyl, or R′ and R″ together with the carbon to which they are attached form a C₃-C₇ cycloalkyl group and m is 0, 1, 2, or 3;
Z¹ is S(O)₂, S(O), or C(O); and
Z² is C₁-C₄ alkyl, NR¹R², aryl, arylamino, aralkyl, aralkoxy, or heteroaryl,
R¹ and R² are each independently selected from hydrogen, C₁-C₄ alkyl, C₃-C₇ cycloalkyl, —S(O)₂R³, and —C(O)R³; and
R³ is C₁-C₄ alkyl or C₃-C₇ cycloalkyl.

3. A process for preparing a compound of formula (I) 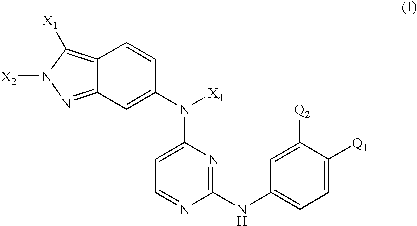
comprising the steps of:
(i) reacting a compound of formula (Q′) 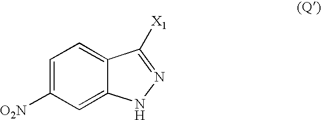
with an alkylating agent to prepare a compound of formula (R′), 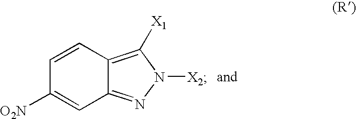
(ii) converting the compound of formula (R′) to the compound of formula (I), said converting step comprising serial condensation with a compound of formula (A′) and then a compound of formula (A″) 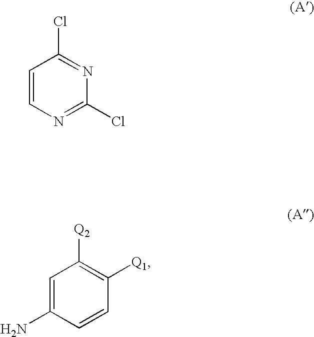
wherein:
X₁ is hydrogen or C₁-C₄ alkyl;
X₂ is C₁-C₄ alkyl or benzyl;
X₄ is hydrogen or C₁-C₄ alkyl;
Q₁ is A¹ or A²;
Q₂ is A¹ when Q₁ is A² and Q₂ is A² when Q₁ is A¹;
wherein
A¹ is hydrogen, halogen, C₁-C₃ alkyl, C₁-C₃ haloalkyl, C₁-C₄ alkoxy, and
A² is the group defined by -(Z)ₘ-(Z¹)-(Z²), wherein
Z is C(R′)(R″), where R′ and R″ are independently selected from —H or C₁-C₄ alkyl, or R′ and R″ together with the carbon to which they are attached form a C₃-C₇ cycloalkyl group and m is 0, 1, 2, or 3;
Z¹ is S(O)₂, S(O), or C(O); and
Z² is C₁-C₄ alkyl, NR¹R², aryl, arylamino, aralkyl, aralkoxy, or heteroaryl, 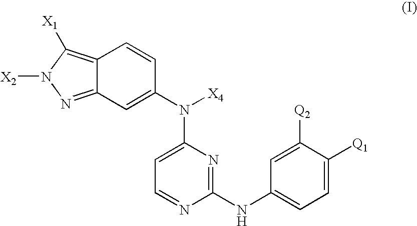
R¹ and R² are each independently selected from hydrogen, C₁-C₄ alkyl, C₃-C₇ cycloalkyl, —S(O)₂R³, and —C(O)R³; and
R³ is C₁-C₄ alkyl or C₃-C₇ cycloalkyl.


SEQUENCE LISTING (1 sequence)

SEQ ID NO: 1 - protein, 14 aa, Artificial Sequence
  hVEGFR2 ligand peptide
EEEEYFELVAKKKK